(19) United States
(12) Patent Application Publication
(10) Pub. No.: US 2006/0216236 A1
(43) Pub. Date: Sep. 28, 2006

(54) Methods of inhibiting alphavBeta3-mediated angiogenesis and tumor development

(76) Inventors: Peter C. Brooks, Carmel, NY (US); Abebe Akalu, Forest Hills, NY (US); Alexandra Cretu, Wilmington, DE (US); Desiree Policarpio, Douglaston, NY (US)

Correspondence Address: WILSON SONSINI GOODRICH & ROSATI, 650 PAGE MILL ROAD, PALO ALTO, CA, 94304-1050, US

(21) Appl. No.: 11/371,620

(22) Filed: Mar. 9, 2006

Related U.S. Application Data
(60) Provisional application No. 60/660,889, filed on Mar. 11, 2005.

(51) Int. Cl. A61K 51/00; C12Q 1/68; G01N 33/574; A61K 39/395

(52) U.S. Cl. 424/1.49; 424/155.1; 424/178.1; 514/2; 435/6; 435/7.23

(57) ABSTRACT

The invention provides methods for identifying genes and proteins modulated by an antagonist of αvβ3 that inhibits binding of αvβ3 to an ECM component. It additionally provides methods for using the products of the identified genes, or for using the identified proteins, for inhibiting angiogenesis, tumor metastasis, and other tumor developmental processes, including cell migration, cell adhesion, cell proliferation, and tumor growth and for treating angiogenesis-dependent conditions. The present invention also relates to antagonists of αvβ3, wherein binding of these antagonists to αvβ3 results in modulation of the expression of IGFBP-4 or TSP-1, and methods of using these antagonists for inhibiting angiogenesis, tumor metastasis, and other tumor development processes as well as for treating angiogenesis-dependent conditions.

### RELATED APPLICATIONS

This application claims the benefit of U.S. Provisional Application No. 60/660,889, entitled “METHODS OF INHIBITING αvβ3 MEDIATED ANGIOGENESIS AND TUMOR DEVELOPMENT,” filed Mar. 11, 2005, by Peter Brooks et al, which is incorporated herein by reference in its entirety.

### REFERENCE TO GOVERNMENT GRANT

This invention was made, in part, with government support under NIH RO1 CA91645 awarded by the National Institutes of Health. The government has rights to the invention.

### FIELD OF THE INVENTION

The present invention relates to the field of medicine, specifically to methods and compositions for inhibiting angiogenesis and other processes important in tumor metastasis, based on identifying genes that are modulated by inhibition of binding of αvβ3-integrin to extracellular matrix components.

### BACKGROUND OF THE INVENTION

The effective treatment of malignant tumors is impeded by the development of resistance to standard therapeutic modalities as well as metastatic dissemination of tumor cells. Metastasis, or the spread of malignant tumor cells from the primary tumor mass to distant sites, involves a complex series of interconnected events. Understanding the biochemical, molecular, and cellular processes that regulate tumor metastasis are of great importance to treating these tumors. The metastatic cascade is thought to be initiated by a series of biochemical and genetic alterations leading to changes in cell-cell interactions allowing disassociation of cells from the primary tumor mass. These events are followed by local invasion and migration through the proteolytically-remodeled extracellular matrix (ECM) to allow access of the tumor cells to the host circulation. In order to establish secondary metastatic deposits, the malignant cells evade the host immune surveillance, arrest in the microvasculature and extravasate out of the circulation. Finally, circulating tumor cells can adhere to the ECM in a new location, proliferate, and recruit new blood vessels by induction of angiogenesis, thereby forming secondary metastatic foci (Liotta, et al., Cell 1991, 64:327-336; Wyckoff, et al., Cancer Res. 2000, 60:2504-2511; Kurschat, et al., Clin. Exp. Dermatol. 2000, 25:482-489; Pantel, et al., Nat. Rev. Cancer 2004, 4:448-456; Hynes, et al., Cell 2003, 113:821-823; Bashyam, M. D., Cancer 2002, 94:1821-1829).

Identification of proteins involved in tumor cell interactions with the proteolytically-remodeled ECM can provide novel therapeutic targets and treatment strategies for treating malignant tumors. While many studies have confirmed the importance of targeting specific secreted growth factors, proteases, cell surface adhesion receptors and intracellular regulatory molecules, the success of these approaches has been limited due in part to the genetic instability of tumor cells (Molife, et al., Crit. Rev. Oncol. Hematol. 2002, 44:81-102; Brown, et al., Melanoma 2001, 3:344-352; Soengas, et al., Oncogene 2003, 22:3138-3151; Masters, et al., Nat. Rev. Cancer 2003, 3:517-525). Therefore, identifying new functional targets within the non-cellular compartment provides a promising clinical strategy.

The ECM is an interconnected molecular network that not only provides mechanical support for cells and tissues, but also regulates biochemical and cellular processes such as adhesion, migration, gene expression and differentiation. Extracellular matrix components include, e.g., collagen, fibronectin, osteopontin, laminin, fibrinogen, elastin, thrombospondin, tenascin, and vitronectin. Cryptic sites, including HUIV26, within collagen, regulate angiogenesis and endothelial cell behavior (Xu, et al., Hybridoma 2000, 19:375-385; Xu, et al., J. Cell Biol. 2001, 154:1069-1079; Hangai, et al., Am. J. Pathol. 2002, 161:1429-1437; Lobov, et al., Proc. Natl. Acad. Sci. USA 2002, 99:11205-11210). This functional cryptic site was shown to be highly expressed within the ECM of malignant tumors and within the sub-endothelial basement membrane of tumor-associated blood vessels, and its exposure found to be involved in the regulation of angiogenesis in vivo (Xu, et al., Hybridoma 2000, 19:375-385; Xu, et al., J. Cell Biol. 2001, 154:1069-1079; Hangai, et al., Am. J. Pathol. 2002, 161:1429-1437; Lobov, et al., Proc. Natl. Acad. Sci. USA 2002, 99:11205-11210, and U.S. Ser. No. 09/478,977, now U.S. Pub. No. 2003/0113331, the disclosure of which is incorporated herein by reference in its entirety).

Cryptic sites in the ECM component, laminin, have also been described, e.g., in U.S. Publication No. 2004/224896 A1 (the disclosure of which is incorporated herein by reference in its entirety), and WO 2004/087734.

There are potentially important cryptic epitopes in other ECM proteins, e.g., fibronectin (Hocking, et al., J. Cell. Biol. 2002, 158:175-184), fibrinogen (Medved et al., Ann. N.Y. Acad. Sci. 2001, 936:185-204), and osteopontin (Yamamoto, et al., J. Clin. Invest. 2003, 12:181-188).

Angiogenesis is the physiological process by which new blood vessels develop from pre-existing vessels (Vamer, et al., Cell Adh. Commun. 1995, 3:367-374; Blood, et. al., Biochim. Biophys. Acta. 1990, 1032:89-118; Weidner, et al., J. Natl. Cancer Inst. 1992, 84:1875-1887). Angiogenesis has been suggested to play roles in both normal and pathological processes. For example, angiogenic processes are involved in the development of the vascular systems of animal organs and tissues. They are also involved in transitory phases of angiogenesis, for example during the menstrual cycle, in pregnancy, and in wound healing. On the other hand, a number of diseases are known to be associated with deregulated angiogenesis.

In certain pathological conditions, angiogenesis is recruited as a means to provide adequate blood and nutrient supply to the cells within the affected tissue. Many of these pathological conditions involve abberant cell proliferation or regulation. Therefore, inhibition of angiogenesis is a potentially useful approach to treating diseases that are characterized by unregulated blood vessel development. For example, angiogenesis is involved in pathologic conditions including: ocular diseases, e.g., macular degeneration, neovascular glaucoma, retinopathy of prematurity and diabetic retinopathy; inflammatory diseases, e.g., immune and non-immune inflammation, rheumatoid arthritis, osteoarthritis, chronic articular rheumatism and psoriasis; chronic inflammatory diseases, e.g. ulcerative colitis and Crohn's disease; corneal graft rejection; vitamin A deficiency; Sjorgen's disease; acne rosacea; mycobacterium infections; bacterial and fungal ulcers; Herpes simplex infections; systemic lupus; retrolental fibroplasia; rubedsis; capillary proliferation in atherosclerotic plaques, and osteoporosis. Angiogenesis is also involved in cancer-associated disorders, including, for example, solid tumors, tumor metastases, blood borne tumors such as leukemias, angiofibromas, Kaposi's sarcoma, benign tumors such as hemangiomas, acoustic neuromas, neurofibromas, trachomas, and pyogenic granulomas, as well as other cancers which require neovascularization to support tumor growth. Other angiogenesis-dependent conditions include, for example, hereditary diseases such as Osler-Weber Rendu disease and haemorrhagic teleangiectasia; myocardial angiogenesis; plaque neovascularization; hemophiliac joints and wound granulation. Progression of tumors such as melanoma, from benign to metastatic disease, correlates with an increase in angiogenesis as well as an increase in expression of specific cell adhesion receptors including integrins (Srivastava, et al., Am. J. Pathol. 1988, 133:419-423; Koth, et al., N. Engl. J. Med. 1991, 325:171-182). Thus, angiogenesis likely plays a critical role in melanoma progression.

Examples of normal physiological processes involving angiogenesis include embryo implantation, embryogenesis and development, and wound healing. It is conceivable that angiogenesis can also be altered to beneficially influence normal physiological processes. Furthermore, studies have indicated that adipose tissue growth is dependent on angiogenesis, likely due to the need for recruitment of new blood vessels. Delivery of an angiogenesis inhibitor to mice was found to reduce diet-induced obesity, the most common type of obesity in humans (Brakenhielm, et al., Circ. Res. 2004, 94(12):1579-88). This finding suggests utility for angiogenesis inhibitors in addressing obesity and certain related conditions. Therefore, the inhibition of angiogenesis potentially can be applied in normal angiogenic responses where a prophylactic or therapeutic need or benefit exists.

An important group of molecules that mediate cellular interactions with the ECM include the integrin family of cell adhesion receptors. Integrins are a family of heterodimeric cell surface proteins composed of non-covalently associated α and β chains (Jin, et al., Br. J. Cancer. 2004, 90:561-565; Bershadsky, et al., Annu. Rev. Cell Dev. Biol. 2003, 19:677-695, and; Parise, et al., Semin. Cancer Biol. 2003,10:407-414). Integrins not only facilitate physical interactions with the ECM but also play critical roles in bi-directional signaling between the ECM and cells. In this regard, αvβ3 is one of the most well-studied integrins thought to play a critical role in invasive cellular processes such as angiogenesis and tumor invasion (Jin, et al., Br. J. Cancer. 2004, 90:561-565; Bershadsky, et al., Annu. Rev. Cell Dev. Biol. 2003, 19:677-695; Parise, et al., Semin. Cancer Biol. 2003, 10:407-414). In fact, expression of αvβ3 in endothelial cells regulates cell survival and apoptosis by a mechanism that likely depends on P53 (Stromblad, et al., A. Suppression of p53 activity and P21WAF1/CIP1 expression by vascular integrin αvβ3 during angiogenesis. J. Clin. Invest. 1996, 98:426-433; Stromblad, et al., A. Loss of p53 compensates for αv-integrin function in retinal neovascularization. J. Biol. Chem. 2002, 277:13371-13374; Lewis, et al., Integrins regulate the apoptotic response to DNA damage through modulation of P53. Proc. Natl. Acad. Sci. USA. 2002, 99:3627-3632). Therefore, αvβ3 ligation might suppress p53 activity. Furthermore, antagonists of αvβ3 failed to inhibit retinal neovascularization in p53 null mice Stromblad, et al., A. Suppression of p53 activity and P21WAF1/CIP1 expression by vascular integrin αvβ3 during angiogenesis. J. Clin. Invest. 1996, 98:426-433; Stromblad, et al., A. Loss of p53 compensates for αv-integrin function in retinal neovascularization. J. Biol. Chem. 2002, 277:13371-13374). Importantly, studies have indicated that αvβ3 plays a critical role in angiogenesis since antagonists directed to αvβ3 inhibit angiogenesis and tumor growth in multiple models (Brooks, et al., A. Requirement of Vascular Integrin αvβ3 for Angiogenesis. Science 1994, 264:569-571; Brooks, et al., Integrin αvβ3 Antagonists Promote Tumor Regression by Inducing Apoptosis of Angiogenic Blood Vessels. Cell, 1994, 79:1157-1164; Brooks, et al., Antiintegrin αvβ3 Blocks Human Breast Cancer Growth and Angiogenesis in Human Skin. J. Clin. Invest. 1995, 96:1815-1822). However in recent studies, mice lacking expression of αvβ3 exhibited enhanced growth of transplanted tumors (Taverna, et al., Increased primary tumor growth in mice null for beta-3 or beta-3/beta-5 integrins or selectins. Proc. Natl. Acad. Sci. USA. 2001, 101:763-768). Thus, the molecular mechanisms by which αvβ3 regulates angiogenesis and tumor growth are complex and to date are not completely understood. Interestingly, αvβ3 and αvβ5 may regulate angiogenesis induced by distinct growth factors by mechanisms dependent on differential phosphorylation of Raf (Hood, et al., A. Differential αv integrin-mediated ras-erk signaling during two pathways of angiogenesis. J. Cell Biol. 2003, 162:933-943; Alavi, et al., A. Role of raf in vascular protection from distinct apoptotic stimuli. Science 2003, 301:204-206). Moreover, intriguing new studies have provided evidence that integrins can regulate signaling cascades in both the unligated and ligated states (Stupack, et al., Apoptosis of Adherent Cells by Recruitment of Caspase-8 to Unligated Integrins. J. Cell Biol. 2001, 155:459-470). In fact, studies suggest that unligated αvβ3 may lead to induction of apoptosis by a mechanism involving recruitment of caspase-8 (Stupack, et al., Apoptosis of Adherent Cells by Recruitment of Caspase-8 to Unligated Integrins. J. Cell Biol. 2001, 155:459-470). Thus, the ability of αvβ3 to either interact or not with distinct ligands may differentially impact invasive cellular behavior. However, gene modulation resulting from binding of integrins to cryptic epitopes of ECM components has not been characterized or systematically studied.

Proteolytic activity plays a crucial role in controlling angiogenesis by releasing matrix-sequestered growth factors as well as remodeling ECM proteins. While many ECM proteins have been shown to bind to αvβ3 in vitro, the physiological relevance of these interactions is not completely understood. ECM remodeling of the matrix can alter the three-dimensional structure of ECM proteins such as collagen and laminin, thereby exposing cryptic regulatory sites that are recognized by integrins including αvβ3 (Xu, et al., J. Cell Biol. 2001, 154:1069-1079; Hangai, et al., Matrix Metalloproteinase-9-Dependent Exposure of a Cryptic Migratory Control Site in Collagen is Required before Retinal Angiogenesis. Am. J. Pathol. 2002, 161:1429-1437; Xu, et al., Generation of Monoclonal Antibodies to Cryptic Collagen Sites by Using Subtractive Immunization. Hydridoma 2000, 19:375-385). Other ligands, including fibrin, fibrinogen, laminin, thrombospondin, vitronectin, von Willebrand's factor, osteospontin and bone sialoprotein I, also bind to αvβ3. The physiological importance of cellular interactions with these cryptic sites has been suggested, since function-blocking Mabs directed to the HUIV26 cryptic collagen site block angiogenesis and tumor growth in a number of animal models (Xu, et al., J. Cell Biol. 2001, 154:1069-1079; Hangai, et al., Matrix Metalloproteinase-9-Dependent Exposure of a Cryptic Migratory Control Site in Collagen is Required before Retinal Angiogenesis. Am. J. Pathol. 2002, 161:1429-1437; Xu, et al., Generation of Monoclonal Antibodies to Cryptic Collagen Sites by Using Subtractive Immunization. Hydridoma 2000, 19:375-385). The HUIV26 cryptic collagen epitope is recognized by αvβ3 integrin, which is highly expressed in tumor-associated blood vessels. Manipulating the interactions between αvβ3 and ECM components could provide a productive strategy for identifying methods to treat tumor development processes, including, but not limited to, tumor metastasis, tumor growth, angiogenesis, cell migration, cell adhesion, cell proliferation and cell proliferation. However, the genes regulated in response to interactions involving integrin receptors and cryptic ECM components has not been previously characterized, and relatively little is known concerning the potential role of these interactions in tumor development processes.

Other proteins appear to be involved in integrin signaling, for example, Insulin Growth Factor Binding Proteins (IGFBPs). IGFBPs are a family of secreted proteins that function to regulate IGF-signaling by binding to IGFs, thereby disrupting IGF receptor binding and subsequent signaling (Pollak, et al., Nat. Rev. Cancer 2004, 4:505-518; Mohan, et al., J. Endocrinol. 2002, 175:19-31; LeRoith, et al., Cancer Lett. 2003, 195:127-137). Specific IGFBPs may directly bind to integrin receptors, thereby modulating their function independently from IGFs (McCaig, et al., J. Cell Sci. 2002, 115:4293-4303; Schutt, et al., J. Mol. Endocrinol. 2004, 32:859-868; Furstenberger, et al., Lancet. 2002, 3:298-302). IGFBPs may regulate cellular adhesion, migration and tumor growth by both IGF-dependent and independent mechanisms (McCaig, et al., J. Cell Sci. 2002, 115:4293-4303; Schutt, et al., J. Mol. Endocrinol. 2004, 32:859-868; Furstenberger, et al., Lancet. 2002, 3:298-302). However, regulation of these cellular processes by integrin-receptor binding of IGFBPs, and the exact role of IGFBPs in these processes, have not been established.

Molecular alterations that occur in both tumor and stromal cells are thought to potentiate angiogenesis in part by modifying expression and bioavailability of angiogenic growth factors as well as altering expression of matrix-degrading proteases. Collectively, these and other molecular changes help to create a microenvironment conducive to new blood vessel growth, one factor that contributes to metastasis and tumor growth. There is evidence for the importance of numerous molecular regulators that contribute to new blood vessel growth, including matrix-degrading proteases such as MMP-9, angiogenesis inhibitors such as TSP-1 and angiogenic growth factors such as VEGF (see, e.g., Yu, et al., Proc. Natl. Acad. Sci. USA 1999, 96:14517-14522; Dameron, et al., Science 1994, 265:1582-1584). These molecular regulators, the proteins that in turn regulate them, and any of a number of other molecules potentially affect angiogenesis and metastasis. However, the exact mechanisms of the regulation of these and related processes, including the genes and gene expression patterns involved, have not been determined.

Further, the protein Id-1 has been reported to repress TSP-1 expression and regulate angiogenesis in vivo (Volpert, et al., Cancer Cell 2002, 2(6):473-83). P53, a tumor-suppressor protein, has also been reported to play an important role in controlling expression of proteins known to regulate angiogenesis, including VEGF and thrombospondin-1 (TSP-1) (Yu, et al., Proc. Natl. Acad. Sci. USA 1999, 96:14517-14522 and Dameron, et al., Science 1994, 265:1582-1584). The p53 status of tumors is believed to impact the efficacy of anti-angiogenic, chemotherapeutic and radiation therapy for the treatment of malignant tumors (Yu, et al., Science 2002, 295:1526-1528; Martin, et al., Cancer Res. 1999, 59:1391-1399; Fridman, et al., Oncogene 2003, 22:9030-9040; Gudkov, et al., Nat. Rev. Cancer 2003, 3:117-128). Despite the possibility that these and other proteins are involved in the integrin-mediated regulation of tumor development processes, e.g., angiogenesis, metastasis, cell adhesion, cell migration, cell proliferation, and tumor growth, the regulation of specific genes in response to αvβ3 binding of ECM component cryptic epitopes has not been previously characterized. This invention identifies the connection between αvβ3 binding of ECM component cryptic epitopes and the regulation of genes involved in tumor development processes.

### SUMMARY OF THE INVENTION

The present invention relates to the discovery that αvβ3 antagonists that inhibit binding of αvβ3 to cryptic ECM components modulate the expression of genes that affect processes important in angiogenesis and metastasis.

The present invention relates to methods for the identification of at least one gene or protein, wherein the expression of said gene or protein is modulated by binding of an antagonist to αvβ3 and wherein the antagonist of αvβ3 binds to αvβ3 and inhibits binding of αvβ3 to an ECM component. It further relates to methods for inhibiting angiogenesis, tumor metastasis, and related processes, including cell migration, cell adhesion, cell proliferation, tumor growth, angiogenesis, and for treating angiogenesis-dependent conditions, using proteins identified based on the modulation of their expression when an antagonist of αvβ3 binds to αvβ3 and inhibits binding of αvβ3 to an ECM component. The present invention also relates to antagonists of αvβ3, wherein binding of the antagonists to αvβ3 results in modulation of the expression of IGFBP-4 or TSP-1. Further, the invention includes methods for the use of these antagonists to inhibit angiogenesis, metastasis, and related processes, as well as for treatment of angiogenesis-dependent conditions, and methods for detecting the inhibition of these processes and conditions based on modulation of IGFBP-4 and TSP-1.

Specifically, the invention contemplates a method for identifying at least one gene or protein, wherein the expression of said gene or protein is modulated by binding of an antagonist to αvβ3, and wherein said antagonist binds to αvβ3 and inhibits binding of αvβ3 to an ECM-component, comprising the steps of: a) treating cells with the antagonist; b) measuring gene expression or protein levels in the cells; c) comparing said gene expression or protein levels with control gene expression or protein levels measured in cells not treated with the antagonist, and; d) identifying a gene or protein wherein said gene expression or protein levels in the cells treated with the antagonist are modulated as compared to the control cell gene expression or protein levels.

The present invention further contemplates methods for inhibiting tumor metastasis, cell adhesion, cell migration, tumor growth, cell proliferation, angiogenesis, or for treating an angiogenesis-dependent condition comprising administering the product of a gene or a protein, wherein the gene or the protein is modulated by inhibiting αvβ3, wherein the gene is identified using a method for identifying at least one gene or protein that is modulated by binding of an antagonist to αvβ3, and wherein said antagonist binds to αvβ3 and inhibits binding of αvβ3 to an ECM-component, said method for identifying comprising the steps of: a) treating cells with the antagonist; b) measuring gene expression or protein levels in the cells; c) comparing said gene expression or protein levels with control gene expression or protein levels measured in cells not treated with the antagonist, and; d) identifying a gene or protein wherein levels of said gene expression or protein levels in the cells treated with the antagonist are modulated as compared to the control cell gene expression or protein levels. In embodiments of these methods, the gene product or protein is administered in conjunction with chemotherapy, radiation therapy, or a cytostatic agent.

In related embodiments, the invention relates to the above methods wherein at least two genes or proteins are identified, and wherein one of the at least two genes or proteins identified is IGFBP-4 or TSP-1.

In certain embodiments, the antagonist used in the above methods is an antibody or an antibody fragment. In embodiments, the antibody can be a monoclonal antibody or a polyclonal antibody. In specific embodiments, the monoclonal antibody is LM609 (Vitaxin).

In other embodiments, the antagonist used in the above methods is an organic peptidomimetic inhibitor. The invention also contemplates the use of a peptide or polypeptide antagonist in the above methods of the invention.

Other embodiments of the above methods include certain methods wherein the ECM component is collagen, laminin, vitronectin, fibrinogen, and methods wherein the ECM component is denatured or proteolyzed.

The invention also contemplates antagonists that bind to αvβ3, wherein binding of said antagonists inhibits the binding of αvβ3 to an ECM component, and wherein the binding of these antagonists to the ECM component results in modulation of IGFBP-4 or TSP-1. In embodiments of the invention, the antagonist is an antibody or antibody fragment, monoclonal antibody, polyclonal antibody, and in specific embodiments, the monoclonal antibody antagonist is LM609 (Vitaxin). Also contemplated are organic peptidomimetic inhibitor, peptide, and polypeptide antagonists.

In related embodiments, the antagonist inhibits the binding of αvβ3 to the ECM component collagen, fibrin, fibrinogen, laminin, thrombospondin, vitronectin, von Willebrand's factor, osteospontin or bone sialoprotein I. In other related embodiments, the antagonist inhibits the binding of αvβ3 to a denatured or proteolyzed ECM component. The invention also contemplates methods of administering these antagonists to inhibit tumor metastasis, cell adhesion, cell migration, tumor growth, angiogenesis, cell proliferation, and to treat an angiogenesis-dependent condition. Further, the invention contemplates administration of the antagonists in conjunction with a monoclonal αvβ3 antagonist of a cryptic ECM component, chemotherapy, radiation therapy, or a cytostatic agent.

The present invention further relates to methods for detecting the inhibition of tumor metastasis, cell adhesion, cell migration, tumor growth, cell proliferation, and angiogenesis using an antagonist that specifically binds to αvβ3, comprising: measuring the level of IGFBP-4 or TSP-1, wherein said level of IGFBP-4 or TSP-1 is modulated.

The present invention also contemplates methods of diagnosing an angiogenesis-dependent condition wherein modulation of genes identified according to the identifying methods of the invention is indicative of the presence or severity of the condition.

### BRIEF DESCRIPTION OF THE DRAWINGS

FIG. 1 Expression of αvβ3 Enhances Human Melanoma Growth In Vivo. Human melanoma cells expressing (M21) or lacking αvβ3 (M21L) were injected (1×10⁶) subcutaneously into nude mice. Tumor cells were allowed to grow for 7 days. Tumor volumes were calculated using the formula V=L2×W/2 where V=volume, L=length and W=width. Data bars represent mean tumor volumes+standard errors from 5 animals per condition. αvβ3-expressing M21 cells formed tumors that were approximately 9-fold larger (P<0.05) than tumors from cells that lacked αvβ3 (M21L). Experiments were completed 3 times with similar results.

FIG. 2 Isolation of αvβ3 Expression Variants of Human ECV Bladder Carcinoma (Parental Cells). Human ECV304 carcinoma cells were subjected to FACS following incubation with Mab LM609 (anti-αvβ3). Four negative selections for expression of αvβ3 integrin were carried out. The figure shows a histogram of FACS analysis for surface expression of integrins αvβ3 (Mab LM609), β1 (Mab P4C 10) or control (non-specific Ab) in parental ECV carcinoma cells. As shown, the parent ECV carcinoma cells expressed high surface levels of αvβ3 (middle panel) and β1 integrins (bottom panel).

FIG. 3 Isolation of αvβ3 Expression Variants of Human ECV Bladder Carcinoma (Variant Cells). Human ECV304 carcinoma cells were subjected to FACS following incubation with Mab LM609 (anti-αvβ3). Four negative selections for expression of αvβ3 integrin were carried out. The figure shows a histogram of FACS analysis for surface expression of integrins αvβ3 (Mab LM609), β1 (Mab P4C10) or control (non-specific Ab) in negative selected carcinoma cells (ECVL). Negatively-selected (ECVL) cells expressed no detectable αvβ3 on the cell surface (middle panel). Reduction of αvβ3 expression in these cells resulted in little if any change in β1 integrin expression (bottom panel).

FIG. 4 Expression of αvβ3 Enhances Human Carcinoma Growth In Vivo. Human carcinoma cells expressing (ECV) or lacking αvβ3 (ECVL) were injected (1.×10⁶) subcutaneously into nude mice. Tumor cell variants were allowed to grow for 14 days. Tumor volumes were calculated using the formula V=L²×W/2 where V=volume, L=length and W=width. Data bars represent mean tumor volumes±standard errors from 5 animals per condition. αvβ3 expressing ECV cells formed tumors that were approximately 3-fold larger than ECVL cells lacking αvβ3. Experiments were completed 3 times with similar results.

FIG. 5 Expression of αvβ3 Does Not Enhance Human Carcinoma Growth In Vitro. Human carcinoma cells expressing (ECV) or lacking αvβ3 (ECVL) were allowed to proliferate in vitro. Tumor cells (ECV and ECVL) were seeded into microtiter plates and allowed to proliferate in low serum (1.0%) containing medium over a time course of 3 days. Proliferation was quantified by monitoring mitochondrial dehydrogenase activity at 490 nm. Data bars represent mean O.D±standard deviation from triplicate wells. Little if any change in proliferation was detected between ECV and ECVL cells in vitro. Experiments were completed 3 times with similar results.

FIG. 6 Reduced Angiogenesis in Tumors Lacking Integrin αvβ3. Tumor angiogenesis was quantified in tumors expressing (M21 and ECV) or lacking αvβ3 (M21L and ECVL) by microvascular density counts. Frozen sections of tumors were stained with an anti-CD31 polyclonal antibody. The number of CD-31 positive blood vessels was counted per 200× microscopic fields. A). Quantification of tumor angiogenesis in M21 melanoma tumor variants. B). Quantification of tumor angiogenesis in ECV carcinoma variants. Data bars represent the mean blood vessel counts per 200× field (N=10 fields per specimen with 3 specimens per tumor type). The αvβ3-expressing tumors (M21 and ECV) exhibited a significant (P<0.05) 2.0 to 2.5-fold increase in the number of blood vessels as compared to tumors lacking αvβ3 (M21L and ECVL).

FIG. 7 Enhanced Blood Flow in αvβ3-Expressing Tumors. Melanoma tumors expressing (CS1β3) and lacking (CS1) αvβ3 were scanned using a Moor LDI VR laser Doppler. Laser Doppler scans on tumor and tissue 0.5 cm surrounding the tumor were performed. The tumor and surrounding tissue were scanned in a raster pattern and the Doppler shifts within the microvasculature were measured. The figure shows representative scans of flow in color-coded digital images (red represents high flow and blue represents low flow). CS1β3 tumors were associated with elevated levels of blood flow (red color) as compared to CS1 tumors.

FIG. 8 Quantification of Enhanced Blood Flow in αvβ3 Expressing Tumors. Melanoma tumors expressing (CS1β3) and lacking (CS1) αvβ3 were scanned using a Moor LDI VR laser Doppler. Laser Doppler scans on tumor and tissue 0.5 cm surrounding the tumor was performed. The tumor and surrounding tissue was scanned in a raster pattern and the Doppler shifts within the microvasculature were measured. The figure shows quantification of tumor-associated blood flow reported numerically using Moor LDI Imaging Software, v3.09. N=5. CS1β3 tumors were associated with an approximately 40% increase in blood flow as compared to CS1 tumors (P<0.05) that lacked αvβ3.

FIG. 9 Conditioned Medium (CM) from Tumors Cells Lacking αvβ3 Inhibits Angiogenesis. Filter disc-containing bFGF (12 ng) was placed on the CAMs of 10-day old chick embryo. Twenty-four hours later the embryos were treated topically with serum free CM (40 μl). At the end of 3 days, angiogenesis was quantified by counting blood vessel branch points. The figure shows the effects of CM from ECV cells on bFGF-induced angiogenesis. Data bars represent the mean number of blood vessel branch point±standard deviation from 8 to 10 embryos per condition. CM from ECVL cells significantly (P<0.001) inhibited bFGF-induced angiogenesis by greater than 90% as compared to control. CM from ECV cells had no significant effect (P>0.300) on angiogenesis. Experiments were completed twice with similar results.

FIG. 10 Conditioned Medium (CM) from Tumors Cells Lacking αvβ3 Inhibit Endothelial Cell Proliferation. Endothelial cells (HUVECs) were seeded into microtiter plates in the presence or absence of serum free CM (25 μl) from either ECV or ECVL and allowed to proliferate in low serum (5.0%) medium for 24 hours. Proliferation was quantified by monitoring mitochondrial dehydrogenase activity at 490 nm using the WST-1 proliferation kit (Chemicon). Data bars represent mean O.D±standard deviation from triplicate wells. CM from ECVL cells inhibited HUVEC cell proliferation by approximately 50%, while CM from ECV cells had no effect. Experiments were completed 3 times with similar results.

FIG. 11 Conditioned Medium (CM) from Tumor Cells (ECVL) Lacking αvβ3 Inhibits Tumor Growth In Vivo. Tumor cells (CS1) were seeded on the CAMs of 10-day old chick embryos. Twenty-four hours later the embryos were treated daily by topical addition of serum-free CM (25 μl) from ECVL. Tumors were allowed to grow for 7 days, then harvested and wet weights determined. Data bars represent the mean tumor weights±standard deviation from 8 to 10 embryos per condition. Control=serum-free concentrated medium. Daily treatments with CM from ECVL tumor cells resulted in a significant decrease (P<0.05) in tumor weight by approximately 50% as compared to controls. Experiments were completed twice with similar results.

FIG. 12 Conditioned Medium (CM) from Tumor Cells (M21) Lacking αvβ3 Inhibits Tumor Growth In Vivo. Tumor cells (CS1) were seeded on the CAMs of 10-day-old chick embryos. Twenty-four hours later the embryos were treated daily by topical addition of serum-free CM (25 μl) from M21L tumor cells. Tumors were allowed to grow for 7 days, then harvested and wet weights determined. Data bars represent the mean tumor weights±standard deviation from 8 to 10 embryos per condition. Control (serum free concentrated medium). Daily treatments with CM from M21 tumor cells resulted in a significant decrease (P<0.05) in tumor weight by approximately 50% as compared to controls. Experiments were completed twice with similar results.

FIG. 13 Elevated Levels of TSP-1 in CM from Tumor Cells Lacking αvβ3 (ECVL). Concentrated serum-free CM was examined for the relative levels of TSP-1 by ELISA. CM (25 μl) from ECV tumor cells was diluted in coating buffer 1:1 and incubated in microtiter wells for 18 hours at 4° C. The wells were washed, blocked and incubated with anti-TSP-1 Mab or control non-specific antibody. The relative levels of TSP-1 were detected by incubation with HRP-labeled goat anti-mouse antibody. All data were corrected for non-specific binding. Data bars represent the mean O.D±standard deviations from triplicate wells. The relative levels of TSP-1 were found to be increased in CM from ECVL by nearly 4-fold as compared to CM-form ECV. Experiments were completed 3 times with similar results.

FIG. 14 Elevated Levels of TSP-1 in CM from Tumor Cells Lacking αvβ3 (M21L). Concentrated serum-free CM was examined for the relative levels of TSP-1 by ELISA. CM (25 μl) from M21 tumor cells was diluted in coating buffer 1:1 and incubated in microtiter wells for 18 hours at 4° C. The wells were washed, blocked and incubated with anti-TSP-1 Mab or control non-specific antibody. The relative levels of TSP-1 were detected by incubation with HRP-labeled goat anti-mouse antibody. All data was corrected for non-specific binding. Data bars represent the mean O.D±standard deviations from triplicate wells. The relative levels of TSP-1 were found to be increased in CM from M21L by nearly 2-fold as compared to CM form M21. Experiments were completed 3 times with similar results.

FIG. 15 TSP-1-Depleted ECVL CM Fails to Inhibit Endothelial Cell Proliferation. Endothelial cells (HUvECs) were seeded into microtiter plates in the presence or absence of TSP-1 depleted CM or non-specific antibody depleted CM (25 μl) from ECVL cells and allowed to proliferate in low serum (5.0%) containing medium for 24 hours. Proliferation was quantified by monitoring mitochondrial dehydrogenase activity at 490 nm using the WST-1 proliferation kit. Data bars represent mean O.D±standard deviation from triplicate wells. Control-depleted ECVL conditioned medium inhibited HUVEC proliferation by approximately 50% as compared to no treatment. In contrast, CM from ECVL cells that was depleted of TSP-1 exhibited little if any effects on HUVEC cell proliferation. Experiments were completed 2 times with similar results.

FIG. 16 Elevated Levels of IGFBP-4 in Tumor Cells Following siRNA-Mediated Reduction in β3 Integrin. Expression of β3 integrin within M21 and ECV cells was reduced by siRNA. FIG. 16A shows Western blot analysis of β3 integrin or control protein β-Actin in M21 cells transfected with either β3-specific or control-scrambled siRNA. β3 integrin was reduced by greater than 70% in β3 siRNA transfected cells as compared to controls, while no change in β-Actin was observed. FIG. 16B shows Western blot analysis of IGFBP-4 or control protein β-Actin in M21 cells transfected with either β3-specific or a control scrambled siRNA. Expression of IGFBP-4 was increased (>60%) in β3 siRNA transfected cells as compared to control cells.

FIG. 17 Elevated Levels of TSP-1 in Tumor Cells Following siRNA-Mediated Reduction in β3 Integrin. Expression of β3 integrin within M21 and ECV cells was reduced by siRNA. The figure shows real time PCR analysis of TSP-1 expression in ECV cells transfected with either β3 specific or a control scrambled siRNA. The relative levels of TSP-1 were significantly elevated in β3 siRNA transfected ECV cells in which β3 integrin is significantly reduced as compared to control transfected cells.

FIG. 18 TSP-1 Expression Following αvβ3-Integrin-Specific Ligation. Culture plates were coated with either αvβ3 specific ligands (Vitronectin and anti-αvβ3 Mab LM609) or β1 integrin ligands (triple helical collagen type-IV and anti-β1 specific Mab P4C10). M21 cells were allowed to interact with specific ECM proteins. The relative levels of TSP-1 were examined by real time PCR following normalization to non-specific ligand (poly-L lysine). CM from M21 cells interacting with the non-αvβ3 ECM ligand collagen type-IV resulted in an approximately 4-fold increase in TSP-1 as compared to CM from cells interacting with the known αvβ3 ligand vitronectin.

FIG. 19 Suppression of TSP-1 Expression Following αvβ3 Integrin Specific Ligation. Culture plates were coated with either αvβ3 specific ligands (vitronectin and anti-αvβ3 Mab LM609) or β1 integrin ligands (triple helical collagen type-IV and anti-β1 specific Mab P4C10). M21 cells were allowed to interact with specific anti-integrin Mabs. The relative levels of TSP-1 were examined by real time PCR following normalization to non-specific ligand (poly-L lysine). The relative levels of TSP-1 in cells ligating αvβ3 was reduced by greater than 50% as compared to cells ligating β1 integrins as measured by real time PCR.

FIG. 20 Inhibition of αvβ3-Mediated Ligation Increases TSP-1 Expression in M21 Cells. M21 cells were seeded on denatured collagen type-IV coated plates in the presence or absence of anti-αvβ3 specific Mab LM609 or an isotype-matched control antibody. Cells were allowed to incubate for 12 hours in 1.0% serum containing medium. Expression of TSP-1 was examined by real time PCR. Expression levels were normalized for β2 macroglobulin (B2M). The relative level of TSP-1 RNA was elevated by approximately 8-fold in M21 cells treated with the anti-αvβ3 specific Mab LM609 as compared to an isotype-matched control antibody as measured by real time PCR

FIG. 21 Inhibition of αvβ3-Mediated Ligation Increases IGFBP-4 Expression in M21 Cells. M21 cells were seeded on denatured collagen type-IV coated plates in the presence or absence of anti-αvβ3 specific Mab LM609 or an isotype matched control antibody. Cells were allowed to incubate for 12 hours in 1.0% serum containing medium. Expression of IGFBP-4 was examined by RT-PCR. Expression levels were normalized for β2 macroglobulin (B2M). The relative level of IGFBP-4 was elevated by approximately 8-fold in M21 cells treated with the anti-αvβ3 specific Mab LM609 as compared to an isotype matched control antibody.

FIG. 22 Inhibition of αvβ3-Mediated Ligation Increases IGFBP-4 RNA Expression in M21 Tumors. M21 cells were seeded on denatured collagen type-IV coated plates in the presence or absence of anti-αvβ3 specific Mab LM609 or an isotype-matched control antibody. Cells were allowed to incubate for 12 hours in 1.0% serum-containing medium. The figure shows expression of IGFBP-4 in M21 tumors grown in chick embryo, either untreated (NT) or treated systemically with Mab LM609 or control non-specific antibody (100 μg/embryo) N=5. Expression of IGFBP-4 was significantly enhanced in M21 tumors grown in the chick embryo following treatment with Mab LM609.

FIG. 23 Elevated Levels of IGFBP-4 Protein in CM from Tumor Cells Lacking αvβ3. Conditioned Medium (CM) was evaluated for the relative levels of IGFBP-4 by ELISA. The figure shows data obtained using CM (25 μl), from ECV and ECVL tumor cells, diluted in coating buffer 1:1 and incubated in microtiter wells. The wells were washed, blocked and incubated with anti-IGFP-3 and IGFBP-4 Mabs. The relative levels of IGFBP-3 and IGFBP-4 were detected by incubation with HRP-labeled goat anti-mouse antibody. All data were corrected for non-specific binding. Data bars represent the mean O.D±standard deviations from triplicate wells. The relative levels of IGFBP-4 increased in CM from ECVL by greater than 10-fold as compared to ECV, while little if any change in the levels of IGFBP-3 was observed. Experiments were completed 3 times with similar results.

FIG. 24 Elevated Levels of IGFBP-4 Protein in CM from Tumor Cells Lacking αvβ3 as Determined by Western Blotting. CM was examined for the relative levels of IGFBP-4 by Western blot. The figure shows analysis of CM from ECV and ECVL cells, for IGFBP-4, or using soluble fibronectin as control. IGFBP-4 was dramatically increased in the CM of ECVL cells as compared to ECV cells while little or no change was detected in soluble fibronectin.

### DETAILED DESCRIPTION OF THE INVENTION

In describing the present invention, the following terms will be employed, and are intended to be defined as indicated below. Unless otherwise indicated, all terms used herein have the same ordinary meaning as they would to one skilled in the art of the present invention.

Citation of documents herein is not intended as an admission that any of the documents cited herein is pertinent prior art, or an admission that the cited documents are considered material to the patentability of the claims of the present application. All statements as to the date or representations as to the contents of these documents are based on the information available to the applicant and do not constitute any admission as to the correctness of the dates or contents of these documents.

### Definitions

I. Antagonists of Integrin αvβ3

Antagonists of αvβ3 bind to αvβ3 and interfere with functional interactions of αvβ3 with natural αvβ3 ligands. As used herein, the term “antagonists” refers to molecules or compounds including, but not limited to, antibodies, peptides, oligonucleotides, and small molecule compounds. Such antagonists are described in, e.g., U.S. Pat. No. 6,500,924; U.S. Pat. No. 5,753,230; U.S. Pub. No. 2004/0063790 A1; U.S. Pub. No. 2004/0258691; U.S. Pub. No. 2004/0265317; U.S. Pub. No. 2005/0002936, and; U.S. Pub. No. 2004/0176334 (the disclosures of which are incorporated herein by reference in their entirety) as well as in the present application.

II. ECM Components

As used herein, an “ECM component” is a component of the non-cellular compartment. ECM components include, e.g., fibrin, fibrinogen, vitronectin, von Willebrand's factor, osteospontin, bone sialoprotein I, collagen, laminin, elastin, thrombospondin, tenascin, osteopontin, and fibronectin, as well as other proteins and molecules found in association with these ECM components or found in the same location as these ECM components (Gustafsson, E., et al., R. Exp. Cell Res. 2000, 261:52-68; Werb, Z., et al., Ann. N.Y. Acad. Sci. 1998, 857:110-118, and; Heissig, et al., Curr. Opin. Hematol. 2003, 10:136-141).

The methods of the invention contemplate the use of antagonists that inhibit binding of αvβ3 to an ECM component, including cryptic epitopes of ECM components, from any animal. For example, collagens may be from any mammal such as rat, mouse, pig, rabbit etc. or from a bird such as chicken. Generally, a collagen is an extracellular matrix protein containing a [Gly-Xaa-Xaa]ₙ sequence. Collagen types are well known in the art (see, e.g., Olsen, B. R. 1995, Curr. Op. Cell. Biol. 5:720-727; Kucharz, E. J. The Collagens: Biochemistry and Pathophysiology. Springer-Verlag, Berlin, 1992; Kunn, K. in Structure and Function of Collagen Types, eds. R. Mayne and R. E. Burgeson, Academic Press, Orlando; U.S. Pub. No. 2003/0113331). Human collagens are preferred collagens. Denatured collagen refers to collagen that has been treated such that it no longer predominantly assumes the native triple helical form. Denaturation can be accomplished by heating the collagen. In one embodiment, collagen is denatured by heating for about 15 minutes at about 100° C. Denaturation also can be accomplished by treating the collagen with a chaotropic agent. Suitable chaotropic agents include, for example, guanidinium salts. Denaturation of a collagen can be monitored, for example, by spectroscopic changes in optical properties such as absorbance, circular dichroism or fluorescence of the protein, by nuclear magnetic resonance, by Raman spectroscopy, or by any other suitable technique. Denatured collagen refers to denatured full-length collagens as well as to fragments of collagen. A fragment of collagen can be any collagen sequence shorter than a native collagen sequence. For fragments of collagen with substantial native structure, denaturation can be effected as for a native full-length collagen. Fragments also can be of a size such that they do not possess significant native structure or possess regions without significant native structure of the native triple helical form. Such fragments are denatured all or in part without requiring the use of heat or of a chaotropic agent. The term denatured collagen encompasses proteolyzed collagen. Proteolyzed collagen refers to a collagen that has been fragmented through the action of a proteolytic enzyme. In particular, proteolyzed collagen can be prepared by treating the collagen with a metalloproteinase, such as MMP-1, MMP-2 or MMP-9, or by treating the collagen with a cellular extract containing collagen degrading activity. Proteolyzed collagen can also be that which occurs naturally at sites of ECM remodeling in a tissue.

Laminins are a large family of extracellular matrix glycoproteins. Laminins have been shown to promote cell adhesion, cell growth, cell migration, cell differentiation, neurite growth, and to influence the metastatic behavior of tumor cells (U.S. Pat. No. 5,092,885). Laminin, of which there are at least ten isoforms, is a major component of basement membranes and has been shown to mediate cell-matrix attachment, gene expression, tyrosine phosphorylation of cellular proteins, and branching morphogenesis (Streuli, et al., J. Cell Biol. 1993, 129:591-603; Malinda and Kleinman, Int. J. Biochem. Cell Biol. 1996, 28:957-1959; Timpl and Brown, Matrix Biol. 1994, 14:275-281; Tryggvason, Curr. Op. Cell Biol. 1993 5:877-882; Stahl, et al., J. Cell Sci. 1997, 110:55-63). Laminin binds to type IV collagen, heparin, gangliosides, and cell surface receptors and promotes the adhesion and growth of various epithelial and tumor cells as well as neurite outgrowth. Laminin is thought to mediate cell-matrix interactions and to be a structural component of all basement membranes binding to collagen IV, heparin sulfate proteoglycan, and nidogen-entactin. The laminin molecule is composed of three polypeptide chains (α, β, and γ) assembled into a cross-shaped structure. Different α, β, and γ chains may be combined, which accounts for the large size of the laminin family (Jones, J. C. R. et al., Micr. Res. Tech. 2000, 51:211-213; Patarroyo, M. et al., Semin. Cancer Biol. 2002, 12:197-207).

An epitope is that amino acid sequence or sequences that are recognized by an antagonist, e.g., an antibody antagonist of the invention. An epitope can be a linear peptide sequence or can be composed of noncontiguous amino acid sequences. An antagonist can recognize one or more sequences, therefore an epitope can define more than one distinct amino acid sequence target. The epitopes recognized by an antagonist can be determined by peptide mapping and sequence analysis techniques well known to one of skill in the art.

A “cryptic epitope of an ECM component” is an epitope of an ECM component protein sequence that is not exposed for recognition within a native ECM component, but is capable of being recognized by an antagonist of a denatured or proteolyzed ECM component. Sequences that are not exposed, or are only partially exposed, in the native structure are potential cryptic epitopes. If an epitope is not exposed, or only partially exposed, then it is likely that it is buried within the interior of the molecule. The sequence of cryptic epitopes can be identified by determining the specificity of an antagonist. Candidate cryptic epitopes also can be identified, for example, by examining the three-dimensional structure of a native ECM component.

III. Angiogenesis and Diseases Potentially Treated by Inhibitors of Angiogenesis

As used herein, the terms “angiogenesis inhibitory,” “angiogenesis inhibiting” or “anti-angiogenic” include vasculogenesis, and are intended to mean effecting a decrease in the extent, amount, or rate of neovascularization. Effecting a decrease in the extent, amount, or rate of endothelial cell proliferation or migration in the tissue is a specific example of inhibiting angiogenesis.

The term “angiogenesis inhibitory composition” refers to a composition which inhibits angiogenesis-mediated processes such as endothelial cell migration, proliferation, tube formation and subsequently leading to the inhibition of the generation of new blood vessels from existing ones, and consequently affects angiogenesis-dependent conditions.

As used herein, the term “angiogenesis-dependent condition” is intended to mean a condition where the process of angiogenesis or vasculogenesis sustains or augments a pathological condition or beneficially influences normal physiological processes. Therefore, treatment of an angiogenesis-dependent condition in which angiogenesis sustains a pathological condition could result in mitigation of disease, while treatment of an angiogenesis-dependent condition in which angiogenesis beneficially influences normal physiological processes could result in, e.g., enhancement of a normal process.

Angiogenesis is the formation of new blood vessels from pre-existing capillaries or post-capillary venules. Vasculogenesis results from the formation of new blood vessels arising from angioblasts which are endothelial cell precursors. Both processes result in new blood vessel formation and are included in the meaning of the term angiogenesis-dependent conditions. The term “angiogenesis” as used herein is intended to include de novo formation of vessels such as that arising from vasculogenesis as well as those arising from branching and sprouting of existing vessels, capillaries and venules.

Examples of diseases in which angiogenesis plays a role in the maintenance or progression of the pathological state are listed herein in the Background of the Invention. Other diseases are known to those skilled in the art and are similarly intended to be included within the meaning of “angiogenesis-dependent condition” and similar terms as used herein.

IV. Cancers, Tumors, and Tissues

The methods of the invention are contemplated for use in treatment of a tumor tissue of a patient with a tumor, solid tumor, a metastasis, a cancer, a melanoma, a skin cancer, a breast cancer, a hemangioma or angiofibroma and the like cancer, and the angiogenesis to be inhibited is tumor tissue angiogenesis where there is neovascularization of a tumor tissue. Typical solid tumor tissues treatable by the present methods include, but are not limited to, tumors of the skin, melanoma, lung, pancreas, breast, colon, laryngeal, ovarian, prostate, colorectal, head, neck, testicular, lymphoid, marrow, bone, sarcoma, renal, sweat gland, and the like tissues. Further examples of cancers treated are glioblastomas.

A tissue to be treated is a retinal tissue of a patient with diabetic retinopathy, macular degeneration or neovascular glaucoma and the angiogenesis to be inhibited is retinal tissue angiogenesis where there is neovascularization of retinal tissue.

Thus, methods which inhibit angiogenesis in a diseased tissue ameliorate symptoms of the disease and, depending upon the disease, can contribute to cure of the disease. In embodiments, the invention contemplates inhibition of angiogenesis in a tissue. The extent of angiogenesis in a tissue, and therefore the extent of inhibition achieved by the present methods, can be evaluated by a variety of methods, such as are described herein.

Any of a variety of tissues, or organs comprised of organized tissues, can support angiogenesis in disease conditions including skin, muscle, gut, connective tissue, joints, bones and the like tissue in which blood vessels can invade upon angiogenic stimuli. Thus, in one embodiment, a tissue to be treated is an inflamed tissue and the angiogenesis to be inhibited is inflamed tissue angiogenesis where there is neovascularization of inflamed tissue. In this class the method contemplates inhibition of angiogenesis in arthritic tissues, such as in a patient with chronic articular rheumatism, in immune or non-immune inflamed tissues, in psoriatic tissue and the like.

In the absence of neovascularization of tumor tissue, the tumor tissue does not obtain the required nutrients, slows in growth, ceases additional growth, regresses and ultimately becomes necrotic resulting in killing of the tumor. The present invention provides for a method of inhibiting tumor neovascularization by inhibiting tumor angiogenesis according to the present methods. Similarly, the invention provides a method of inhibiting tumor growth by practicing the angiogenesis-inhibiting methods.

The methods are also particularly effective against the formation of metastases because their formation requires vascularization of a primary tumor so that the metastatic cancer cells can exit the primary tumor and their establishment in a secondary site requires neovascularization to support growth of the metastases.

The invention also contemplates the practice of the method in conjunction with other therapies such as conventional chemotherapy directed against solid tumors and for control of establishment of metastases. The administration of an angiogenesis inhibitor is typically conducted during or after chemotherapy, although it is preferable to inhibit angiogenesis after a regimen of chemotherapy at times where the tumor tissue will be responding to the toxic assault by inducing angiogenesis to recover by the provision of a blood supply and nutrients to the tumor tissue. In addition, it is preferred to administer the angiogenesis inhibition methods after surgery where solid tumors have been removed as a prophylaxis against metastases.

V. Patients

The invention contemplates treatment of patients including human patients. The term patient as used in the present application refers to all different types of mammals including humans and the present invention is effective with respect to all such mammals. The present invention is effective in treating any mammalian species which have a disease associated with angiogenesis or which reduction of angiogenesis would result in treatment of a condition including tumor metastasis, tumor growth, cell adhesion, cell proliferation or cell migration. The present invention has particular application to agricultural and domestic mammalian species.

### Modes of Carrying Out the Invention

It is to be understood that this invention is not limited to particular formulations or process parameters, as these may, of course, vary. It is also to be understood that the terminology used herein is for the purpose of describing particular embodiments of the invention only, and is not intended to be limiting. Further, it is understood that a number of methods and materials similar or equivalent to those described herein can be used in the practice of the present invention.

VI. Antagonists of αvβ3

Suitable αvβ3 antagonists used in the present methods are compounds that interfere with functional interactions of αvβ3 with natural αvβ3 ligands. Methods for preparing and identifying certain candidate antagonists of the invention are described in, e.g., U.S. Pat. No. 6,500,924; U.S. Pub. No. 2004/0063790 A1; U.S. Pub. No. 2004/0258691; U.S. Pub. No. 2004/0265317; U.S. Pub. No. 2005/0002936, and; U.S. Pub. No. 2004/0176334.

The present invention contemplates as examples of useful antagonist analogs of αvβ3 which are derived from the portion of αvβ3 that is considered to be the ligand binding site, αvβ3 mimetics, mimetics of natural ligand of αvβ3 that include or functionally act as the structural region involved in the αvβ3-ligand binding, sequences corresponding to the functional binding domain of the αvβ3 natural ligand including peptides and polypeptides, sequences corresponding to the RGD domain of the natural ligand which bond to αvβ3 including peptides, polypeptides and the like, and antibodies monoclonal antibodies which bind with αvβ3 or the natural αvβ3 ligand.

The useful antagonists of αvβ3 have the ability to substantially inhibit the binding of a naturally occurring ligand such as vitronectin or fibrinogen to the αvβ3 molecules. At a concentration of less than 5 μm concentrations less than 0.1 μm, and concentrations of less than 0.05 μm. The term “substantially” indicates that at least 50% of the binding of fibrinogen is reduced in a presence of the αvβ3 antagonist. The term “IC₅₀ value” as used herein is meant to refer to 50% inhibition in binding.

An αvβ3 antagonist may potentially show selective binding to αvβ3 as compared to the binding to other integrins. When an αvβ3 antagonist does show selectivity the binding of αvβ3 to fibrinogen is substantially inhibited but the binding between αvβ3 and other integrins, such a αvβ1, αvβ5, αIIβ3 is not substantially inhibited. The αvβ3 antagonist are particularly useful in the present invention to show a 10-fold to a 100-fold lower IC₅₀ value for inhibiting the binding of αvβ3 to fibrinogen when compared to the IC₅₀ value for binding of αvβ3 to other integrins. The methods for measuring IC₅₀ activity are well known in the art and for example methods for demonstrating inhibitions of fibronecting to a particular integrin is now described in the United States Patent Publication No. 2004/0063790.

VI.A. Peptide and Polypeptide Antagonists of αvβ3

The peptides useful in the present invention can be either linear or cyclical although cyclic peptides are preferred in some applications. Peptides or polypeptides are in longer length, such as a length of greater than 100 amino acid residue, can be produced as a fusion protein or a fragment of a protein as described in the description of this invention. Peptides and polypeptides that are useful in this invention may not have the identical amino acid residue sequences of αvβ3 natural ligand, and it may have that amino acid sequence as part of a longer sequence or a fusion protein as long as that polypeptide or peptide is able to function as a αvβ3 antagonist in the assays useful in this invention.

Polypeptides and peptides of the present invention include any fragment, analog or chemical derivative of that peptide or polypeptide that has an amino acid residue sequence as shown in this application, and that the particular amino acid residue sequence, fragment or chemical derivative functions as a αvβ3 antagonist. The peptides and polypeptides useful in the present invention may include changes, substitutions, insertions and deletions where the changes in the sequence or particular chemical makeup of particular residues provide for certain advantages in the present invention. An αvβ3 antagonist polypeptide or peptide useful in this invention need not be identical to but rather may correspond to the sequence of a particular peptide or polypeptide that is recited in the present application where changes made to that polypeptide or peptide between the αvβ3 antagonist function in an assay described herein.

The polypeptides or peptides of the present invention may be a peptides or polypeptides derivative that include those residue or chemical changes including amides, conjugates with proteins, cyclic peptides, polymerized peptides and analogs of fragments of chemically modified peptides or proteins and other types of derivatives.

As used herein, the term “analog” includes peptides and polypeptides having a sequence of amino acid residues that is substantially identical to an amino acid sequence specifically described in this application in which one or more amino acids has been conservatively substituted with an amino acid residue that functions in a similar manner and allows the resulting αvβ3 antagonist to have the activity described in this application. Conservative substitutions are well known in the art and include the substitutions one non-polar (hydrophobic) residue such as isoleucine, valine, leucine or methionine for another, the substitution of a polar (hydrophilic) residue for another such as the substitution of arginine and lysine, glutamine and asparagine, lysine and serine or a substitution of a basic residue for another basic residue such as lysine, arginine or histidine substitutions. Other conservative substitutions would include the substitutions of acidic amino acid residues for another such as the substitution of aspartic acids or glutamic acid.

The term “conservative substitution” as used in this application includes chemically derivatized residues that are used to replace a non-derivatized residue in a peptide or polypeptide that results in a peptide or polypeptide that maintains the desired function.

The term “chemical derivative” as used in this application refers to polypeptide or peptide having amino acid sequence residues that are changed or derivatized chemically by using a reaction with a functional side group. Other contemplated derivitizations of peptides or polypeptides includes a chemical derivative which uses backbone modifications including α-amino acids substitutions, such as N-methyl, N-ethyl, N-propyl and other similar substitutions to replace various residues within the backbone. Other potential derivatives utilizing backbone modifications include α-carbonyl substitutions such as thioester, thioamide, guanidino, and other similar substitutions. The present invention also contemplates the use of derivitized molecules which include pre-amino acid groups which have been derivitized to form hydroclorides, p-toleune sulfonyl groups carbobenzoxy groups, t-butyloxycarbonyl groups, chloroacetyl groups or formyl groups. The free carboxyl groups typically may be derivitized to form salts, methyl and ethyl esters or other types of esters or hydrazides. The free hydroxyl groups may be derivatized to form O-acyl or O-alkyl derivatives of those peptides or polypeptides.

Solid matrices, labels and carriers that can be used with the polypeptides of this invention are described herein below.

Amino acid residue linkers are usually at least one residue and can be 40 or more residues, more often 1 to 10 residues. Tyrosine, cysteine, lysine, glutamic and aspartic acid are some examples of amino acid residues which are typically used for linking. In addition, a subject polypeptide can differ, unless otherwise specified, from the sequence of an αvβ3 ligand by modifying the sequence with terminal-NH₂ acylation, e.g., acetylation, or thioglycolic acid amidation, by terminal-carboxylamidation, e.g., with ammonia, methylamine, and the like terminal modifications. It is well known that terminal modifications are useful to reduce susceptibility by proteinase digestion, and therefore serve to prolong the half-life of the polypeptides in solutions and in particular in biological fluids where proteases may be present. In this regard, polypeptide cyclization is also a useful terminal modification in view of the biological activities observed for such cyclic peptides and because of the stable structures formed by cyclization.

A peptide of the present invention may be used in the form of a pharmaceutically acceptable salt. Suitable acids which are capable of forming salts with the peptides of the present invention include inorganic acids such as trifluoroacetic acid (TFA) hydrochloric acid (HC), hydrobromic acid, perchloric acid, nitric acid, thiocyanic acid, sulfuric acid, methane sulfonic acid, acetic acid, phosphoric acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid or the like. HCl and TFA salts are particularly preferred.

Suitable bases capable of forming salts with the peptides of the present invention include inorganic bases such as sodium hydroxide, ammonium hydroxide, potassium hydroxide and the like; and organic bases such as mono-, di- and tri-alkyl and aryl amines (e.g. triethylamine, diisopropyl amine, methyl amine, dimethyl amine) and optionally substituted ethanolamines (e.g. ethanolamine and diethanolamine).

In addition, a peptide useful in the methods of this invention can be prepared without including a free ionic salt in which the charged acid or base groups present in the amino acid residue side groups (e.g., Arg, Asp, and the like) associate and neutralize each other to form an “inner salt” compound.

A peptide of the present invention can be synthesized by any of the techniques that are known to those skilled in the art, including polypeptide and recombinant DNA techniques. Synthetic chemistry techniques, such as a solid-phase Merrifield-type synthesis can be advantageous since they produce products having high purity, antigenic specificity, freedom from undesired side products, ease of production and the like. Summaries of the some techniques available can be found in, e.g., Steward et al., “Solid Phase Peptide Synthesis,” W. H. Freeman Co., San Francisco, 1969; Bodanszky, et al., “Peptide Synthesis,” John Wiley & Sons, Second Edition, 1976; J. Meienhofer, “Hormonal Proteins and Peptides,” Vol. 2, p. 46, Academic Press (New York), 1983; Merrifield, Adv. Enzymol. 1969, 32:221-96; Fields et al., Int. J. Peptide Protein Res. 1990, 35:161-214; U.S. Pat. No. 4,244,946 for solid phase peptide synthesis, and Schroder et al., “The Peptides,” Vol. 1, Academic Press (New York), 1965 (for classical solution synthesis). Such synthesis can utilize appropriate protective groups which are described in J. F. W. McOmie, “Protective Groups in Organic Chemistry,” Plenum Press, New York, 1973.

Solid-phase synthesis methods generally comprise the sequential addition of one or more amino acid residues or suitably protected amino acid residues to a growing peptide chain. Normally, either the amino or carboxyl group of the first amino acid residue is protected by a suitable, selectively removable protecting group. For amino acids containing a reactive side group (e.g., lysine), a different, selectively removable protecting group is utilized.

In solid phase synthesis, the protected or derivatized amino acid is attached to an inert solid support through its unprotected carboxyl or amino group. The protecting group of the amino or carboxyl group is then selectively removed and the next amino acid in the sequence having the complimentary (amino or carboxyl) group suitably protected is admixed and reacted under conditions suitable for forming the amide linkage with the residue already attached to the solid support. The protecting group of the amino or carboxyl group is then removed from this newly added amino acid residue, and the next suitably protected amino acid is then added, and so forth. After all the desired amino acids have been linked in the proper sequence, any remaining terminal and side group protecting groups (and solid support) are removed sequentially or concurrently, to afford the final linear polypeptide.

Linear polypeptides may be reacted to form their corresponding cyclic peptides. A method for preparing a cyclic peptide is described by Zimmer et al., Peptides 1992, pp. 393-394, ESCOM Science Publishers, B.V., 1993. Typically, tertbutoxycarbonyl protected peptide methyl ester is dissolved in methanol, sodium hydroxide solution is added, and the admixture is reacted at 20° C. to hydrolytically remove the methyl ester protecting group. After evaporating the solvent, the tertbutoxycarbonyl protected peptide is extracted with ethyl acetate from acidified aqueous solvent. The tertbutoxycarbonyl protecting group is then removed under mildly acidic conditions in dioxane cosolvent. The unprotected linear peptide with free amino and carboxy termini so obtained is converted to its corresponding cyclic peptide by reacting a dilute solution of the linear peptide, in a mixture of dichloromethane and dimethylformamide, with dicyclohexylcarbodiimide in the presence of 1-hydroxybenzotriazole and N-methylmorpholine. The resultant cyclic peptide is then purified by chromatography.

Cyclic peptide synthesis can be achieved by alternative methods as described by Gurrath et al., Eur. J. Biochem. 1992, 210:911-921.

In addition, the antagonist can be provided in the form of a fusion protein. Fusion proteins are proteins produced by recombinant DNA methods known and described in the art, in which the subject polypeptide is expressed as a fusion with a second carrier protein such as a glutathione sulfhydryl transferase (GST) or other well-known carrier.

Thus, a polypeptide can be present in any of a variety of forms of peptide derivatives, including amides, conjugates with proteins, cyclized peptides, polymerized peptides, analogs, fragments, chemically modified peptides, and like derivatives.

Specific peptides and derivative αvβ3 antagonist peptides contemplated as candidates for use in the present invention, including polypeptides derived from MMP-2, are disclosed in U.S. Pub. No. 2003/0176334.

A polypeptide (peptide) αvβ3 antagonist can have the sequence characteristics of the natural ligand of αvβ3. Alternatively, the αvβ3 antagonist can have the sequence characteristics of αvβ3 itself at the region involved in αvβ3-ligand interaction and display αvβ3 antagonist activity as described herein. An αvβ3 antagonist peptide can contain the RGD tripeptide and correspond in sequence to the natural ligand in the RGD-containing region.

Polypeptides can have a sequence corresponding to the amino acid sequence of the RGD-containing region of a natural ligand of αvβ3 such as fibrinogen, vitronectin, von Willebrand factor, laminin, thrombospondin, and the like. The sequence of these αvβ3 ligands are well-known. Thus, an αvβ3 antagonist peptide can be derived from any of the natural ligands.

VI.B. Antibody Antagonists of αvβ3

Polyclonal or monoclonal αvβ3 antagonists in the form of antibodies that immunoreact with αvβ3 and inhibit αvβ3 binding to its natural ligand are contemplated for use in embodiments of the present invention. Antibodies, whether polyclonal or monoclonal, can be raised against the desired proteins or peptides by any methods known in the art (see e.g., Antibody Production: Essential Techniques, Delves, Wiley, John & Sons, Inc., 1997; Basic Methods in Antibody Production and Characterization, Howard and Bethell, CRC Press, Inc., 1999; and Monoclonal Antibody Production Techniques and Applications: Hybridoma Techniques, Schook, Marcel Dekker, 1987).

Particular monoclonal antibodies of this invention immunoreact with isolated αvβ3, and inhibit ECM component binding to αvβ3. Preferred monoclonal antibodies which preferentially bind to αvβ3 include a monoclonal antibody having the immunoreaction characteristics of Mab LM609 (Vitaxin®), secreted by hybridoma cell line ATCC HB 9537. Mab LM609 has been described previously, e.g. in U.S. Publication No. 2005/0002936. Briefly, the monoclonal antibody LM609 secreted by hybridoma ATCC HB 9537 was produced using standard hybridoma methods by immunization with isolated αvβ3 adsorbed onto Sepharose-lentil lectin beads. The αvβ3 had been isolated from human melanoma cells designated M21, and antibody was produced as described by Cheresh et al., J. Biol. Chem., 262:17703-17711 (1987). M21 cells were provided by Dr. D. L. Morton (University of California at Los Angeles, Calif.) and grown in suspension cultures in RPMI 1640 culture medium containing 2 mM L-glutamine, 50 mg/ml gentamicin sulfate and 10% fetal calf serum. Monoclonal antibody LM609 has been shown to immunoreact with the αvβ3 complex, and not immunoreact with αv subunit, with β3 subunit, or with other integrins. The hybridoma cell line ATCC HB 9537 was deposited pursuant to the Budapest Treaty requirements with the American Type Culture Collection (ATCC), 1301 Parklawn Drive, Rockville, Md. USA, on Sep. 15, 1987.

As used in this application the term “antibody” or “antibody molecule” or “antibody fragment” refers to a population of a immunoglobulin molecules and/or immunological active portions of those particular immunoglobin molecules that contain the portion of an antibody which binds to its antigens, also known as the “antibody-combining site.”

The term “antibody” also includes molecules which have been engineered through the use of molecular biological technique to include only portions of the native molecule as long as those molecules have the ability to bind to a particular antigen with the required specification. Such alternative antibody molecules include classically known portions of the antibodies molecules and single chain antibodies.

Antibodies for use in the present invention are intact immunoglobulin molecules, substantially intact immunoglobulin molecules and those portions of an immunoglobulin molecule that contain the paratope, including those portions known in the art as Fab, Fab′, F(ab′)₂, scFv and F(v), also referred to as antibody fragments.

The invention embodies a truncated immunoglobulin molecule comprising a Fab fragment derived from a monoclonal antibody of this invention. The Fab fragment, lacking Fc receptor, is soluble, and affords therapeutic advantages in serum half life and diagnostic advantages in modes of using the soluble Fab fragment. The preparation of a soluble Fab fragment is generally known in the immunological arts and can be accomplished by a variety of methods.

For example, Fab and F(ab′)₂ portions (fragments) of antibodies are prepared by proteolysis using papain and pepsin, respectively, on substantially intact antibodies by methods that are well known. See for example, U.S. Pat. No. 4,342,566 to Theofilopolous and Dixon. Fab′ antibody portions also are well known and are produced from F(ab′)₂ portions, followed by reduction of disulfide bonds linking the two heavy chains as with mercaptoethanol, and followed by alkylation of the resulting protein mercaptan with a reagent such as iodoacetamide.

The term “monoclonal antibody” as used herein refers to an antibody molecule population that has only one particular antibody combining site and is capable of immunoreacting with a particular epitope. A monoclonal antibody typically displays a single binding affinity for that epitope and such binding can be measured by standard amino acids. Monoclonal antibodies that are useful in this invention may also contain a number of different antibody combining sites wherein each antibody combining site is specific for a particular epitope. Examples of such monoclonal antibodies include biospecific monoclonal antibodies. Monoclonal antibodies contemplated by the present invention also include monoclonal antibodies that are produced by various methods including traditional monoclonal antibodies technology and modern molecular techniques which isolate the antibody combining site of a particular antibody and express it as either a part of a immunological molecule or as part of another molecule.

A monoclonal antibody can be composed of antibodies produced by clones of a single cell called a hybridoma that produces only one kind of antibody molecule. The hybridoma cell is formed by fusing an antibody-producing cell and a myeloma or other self-perpetuating cell line. The preparation of such antibodies was first described by Kohler and Milstein, Nature 1975, 256:495-497. Additional methods are described by Zola, Monoclonal Antibodies: A Manual of Techniques, CRC Press, Inc. (1987).

A monoclonal hybridoma culture is initiated comprising a nutrient medium containing a hybridoma that secretes antibody molecules of the appropriate specificity. The culture is maintained under conditions and for a time period sufficient for the hybridoma to secrete the antibody molecules into the medium. The hybridoma supernatant so prepared can be screened for the presence of antibody molecules that immunoreact with αvβ3.

To form the hybridoma from which the monoclonal antibody is produced, a myeloma or other self-perpetuating cell line is fused with lymphocytes obtained from the spleen of a mammal hyperimmunized with a source of αvβ3.

It is preferred that the myeloma cell line used to prepare a hybridoma be from the same species as the lymphocytes. A mouse of the strain 129 G1X⁺ is typically the preferred mammal. Suitable mouse myelomas for use in the present invention include the hypoxanthine-aminopterin-thymidine-sensitive (HAT) cell lines P3x63-Ag8.653, and Sp2/0-Ag14 that are available from the American Type Culture Collection, Rockville, Md., under the designations CRL 1580 and CRL 1581, respectively.

Splenocytes are typically fused with myeloma cells using a space inhibitor such as polyethylene glycol (PEG) 1500. Fused hybrids are selected by their sensitivity to a selective growth medium, such as HAT (hypoxanthine aminopterin thymidine) medium. Hybridomas producing a monoclonal antibody of this invention can be identified using the enzyme linked immunosorbent assay (ELISA).

Media useful for the preparation of these compositions are both well known in the art and commercially available and include synthetic culture media, media derived from inbred mice and the like. An exemplary synthetic medium is Dulbecco's minimal essential medium (DMEM; Dulbecco et al., Virol. 1959, 8:396, 1959) supplemented with 4.5 g/L glucose, 20 nM glutamine, and 20% fetal calf serum. An exemplary inbred mouse strain is the Balb/c.

Alternatively, the monoclonal antibody may be produced using cloning methods to isolate the gene(s) encoding the monoclonal antibody. Such techniques are well known in the art. See, for example, the method of isolating monoclonal antibodies from an immunological repertoire as described by Sastry et al., Proc. Natl. Acad. Sci. USA 1989, 86:5728-5732; and Huse et al., Science 1989, 246:1275-1281.

Antibodies, whether polyclonal or monoclonal, can be raised against the desired proteins or peptides by any methods known in the art (see e.g., Antibody Production: Essential Techniques, Delves, Wiley, John & Sons, Inc., 1997; Basic Methods in Antibody Production and Characterization, Howard and Bethell, CRC Press, Inc., 1999; and Monoclonal Antibody Production Techniques and Applications: Hybridoma Techniques, Schook, Marcel Dekker, 1987).

Humanized monoclonal antibodies offer advantages over murine monoclonal antibodies, particularly insofar as they can be used therapeutically in humans. Human antibodies are not cleared from the circulation as rapidly as “foreign” antigens, and do not activate the immune system in the same manner as foreign antigens and foreign antibodies. Methods of preparing “humanized” antibodies are known in the art, and can be applied to the antibodies of the present invention.

Thus, the invention contemplates, in one embodiment, a monoclonal antibody of this invention that is humanized by grafting to introduce components of the human immune system without substantially interfering with the ability of the antibody to bind antigen.

The antibody of the invention can also be a fully human antibody such as those generated, for example, by selection from an antibody phage display library displaying human single chain or double chain antibodies such as those described in de Haard, H. J. et al., J. Biol. Chem. 1999, 274:18218-30 and in Winter, G. et al., Annu. Rev. Immunol. 1994, 12:433-55.

VI.C. Other Antagonists of αvβ3

Antagonists of the invention also can be small organic molecules, such as those natural products, or those compounds synthesized by conventional organic synthesis or combinatorial organic synthesis. Compounds can be tested for their ability to bind to a αvβ3 for example by using the affinity-purification technique described herein.

Antagonists of the invention also can be non-peptidic compounds, including, for example, oligonucleotides. Oligonucleotides, as used herein, refer to any heteropolymeric material containing purine, pyrimidine and other aromatic bases. DNA and RNA oligonucleotides are suitable for use with the invention, as are oligonucleotides with sugar (e.g., 2′ alkylated riboses) and backbone modifications (e.g. phosphorothioate oligonucleotides). Oligonucleotides may present commonly found purine and pyrimidine bases such as adenine, thymine, guanine, cytidine and uridine, as well as bases modified within the heterocyclic ring portion (e.g., 7-deazaguanine) or in exocyclic positions. “Oligonucleotide” also encompasses heteropolymers with distinct structures that also present aromatic bases, including polyamide nucleic acids and the like.

An oligonucleotide antagonist of the invention can be generated by a number of methods known to one of skill in the art. In one embodiment, a pool of oligonucleotides is generated containing a large number of sequences. Pools can be generated, for example, by solid phase synthesis using mixtures of monomers at an elongation step. The pool of oligonucleotides is sorted by passing a solution containing the pool over a solid matrix to which αvβ3 or fragment thereof has been affixed. Sequences within the pool that bind to the αvβ3 are retained on the solid matrix. These sequences are eluted with a solution of different salt concentration or pH. Sequences selected are subjected to a second selection step. The selected pool is passed over a second solid matrix to which αvβ3 has been affixed. The column retains those sequences that bind to αvβ3, thus enriching the pool for sequences specific for αvβ3. The pool can be amplified and, if necessary, mutagenized and the process repeated until the pool shows the characteristics of an antagonist of the invention. Individual antagonists can be identified by sequencing members of the oligonucleotide pool, usually after cloning said sequences into a host organism such as E. coli.

VII. Identification of Antagonists of αvβ3

Antagonists of αvβ3 have been described in U.S. Publication No. 2003/0113331; U.S. Publication No. 2004/242490 A1; WO 2004/073649; U.S. Publication No. 2004/224896 A1, and; WO 2004/087734. Antagonists are evaluated for their ability to bind αvβ3, and furthermore can be evaluated for their ability to inhibit binding of αvβ3 to an ECM component. Measurement of binding of antagonists to αvβ3, and their ability to inhibit binding of αvβ3 to other molecules, including its natural ligands, can be accomplished, e.g., using an enzyme-linked-immunosorbent assay (ELISA), described in the publications listed above and herein. The ELISA is commonly used and well-known to those of skill in the art.

The ELISA also can be used to identify compounds which exhibit increased specificity for αvβ3 in comparison to other molecules. The specificity assay is conducted by running parallel ELISAs in which a potential antagonist is screened concurrently in separate assay chambers for the ability to bind αvβ3. Another technique for measuring apparent binding affinity familiar to those of skill in the art is a surface plasmon resonance technique (analyzed on a BIACORE 2000 system) (Liljeblad, et al., Glyco. J. 2000, 17:323-329). Standard measurements and traditional binding assays are described by Heeley, R. P., Endocr. Res. 2002, 28:217-229.

Antagonists of αvβ3 can also be identified by their ability to compete for binding with an antagonist useful in the present invention. For example, putative antagonists can be screened by monitoring their effect on the affinity of a known antagonist, such as antibody LM609, described, e.g., in U.S. Publication No. 2005/0002936. Such antagonists likely have the same specificity as, and recognize the same epitope, as the antibody itself. Putative antagonists selected by such a screening method can bind either to αvβ3 or to the known antagonist. Antagonists can be selected from the putative antagonists by conventional binding assays to determine those that bind to αvβ3 but not to the known antagonist.

Antagonists can also be identified by their ability to bind to a solid matrix containing αvβ3. Such putative antagonists are collected after altering solution conditions, such as salt concentration, pH, temperature, etc. The putative antagonists are further identified by their ability to pass through, under appropriate solution conditions, a solid matrix to which αvβ3 has been affixed.

Antagonists useful in the invention also can be assayed for their ability to influence tumor development processes, e.g., angiogenesis, tumor metastasis, cell adhesion, cell migration, cell proliferation, and tumor growth in a tissue. Any suitable assay known to one of skill in the art can be used to monitor such effects. Several such techniques are described herein.

VIII. Methods for Identifying Genes Modulated by Binding of an Antagonist to αvβ3

In methods of the invention, expression of at least one gene or protein is modulated by the binding of an antagonist to αvβ3, wherein the antagonist inhibits binding of αvβ3 to an ECM-component. Methods for identifying modulated genes and proteins of the invention are provided in the examples.

Generally, cells that express αvβ3 and have been associated with an epitope of an ECM component are treated with the antagonist. Association of the epitope of the ECM component and the cells can be accomplished by various means. For example, dishes can be coated with the cryptic epitope and the cells added to the coated dishes. The epitope can also be mixed or contacted with the cells in solution. For example, serum, which contains ECM components including vitronectin and fibronectin, can be added to the cell medium. After antagonist treatment, a comparison of gene expression or protein levels observed in either treated cells or untreated cells is then made. A panel of genes or proteins, or just one gene or protein, can be compared by these methods. Based on analyses of the gene expression or protein levels, modulated genes or proteins can be identified.

As used herein, the term “modulated” is intended to mean either upregulated or downregulated. Modulation of gene expression can be determined by quantitating nucleic acid, e.g., RNA or cDNA, from specific genes. In embodiments, the expression of a gene or protein is upregulated or downregulated at least 1.5-fold, relative to the control gene expression.

For example, as described herein, the relative level of TSP-1 RNA was elevated by approximately 8-fold in M21 cells treated with the αvβ3 specific antagonist, Mab LM609, as compared to an isotype matched control antibody. The relative level of IGFBP-4 was elevated by approximately 8-fold in M21 cells treated with the anti-αvβ3 specific Mab LM609 as compared to an isotype matched control antibody.

Modulation of gene expression levels, including the levels of IGFBP-4 and TSP-1, can be measured using methods well-known to those of skill in the art, e.g., Real Time quantitative RT-PCR. Primer sequences useful for detecting IGFBP-4 and TSP-1 are given below in the Examples, and additional primer sequences for these genes as well as primer sequences for other genes identified as modulated in the methods of the invention can be determined by sequence analysis methods and using software as known and commonly used by those of skill in the art.

In addition to PCR techniques, other methods for detecting and quantifying nucleic acids are well known to those skilled in the art and have been described in the literature, including hybridization methods, e.g., Southern blotting, Northern blotting, etc., with subsequent quantification by known methods. Amplification techniques, including PCR, can be used prior to analysis using one of the above methods.

Expression of test genes can be compared to expression of an internal control gene, e.g., β2-Macroglobulin. Other suitable endogenous internal control genes and methods for identifying control genes in different tissues are well known to those of skill in the art. For example, methods for identifying control genes have been described by Vandesompele, et al., “Accurate normalization of real-time quantitative RT-PCR data by geometric averaging of multiple internal control genes,” Genome Biol. 2002, 3(7): research0034.1-research0034.11.

IX. Methods for Identifying Proteins Modulated by Antagonists of αvβ3

Modulation of protein levels, including those of IGFBP-4 and TSP-1, can be measured using methods described in the literature and well-known to those of skill in the art. Enzyme Linked Immunosorbent Assay (ELISA), Western Blot analysis, radioimmunoassay and immunoprecipitation are examples of methods that can be used to detect and quantitate the proteins of interest. Enzymatic assays, also well known in the art, can also be used where appropriate.

Proteins that are modulated at least 1.5 to 2-fold (up or down) are preferred for use in the methods of the invention. For example, as determined by ELISA and described in Example IX, levels of TSP-1 were found to be increased in conditioned medium (CM) from cells lacking αvβ3 (ECVL and M21L) by nearly 2 to 4 fold as compared to CM from cells expressing αvβ3 (ECV and M21).

As described in Example XV, ELISA showed that the relative levels of IGFBP-4 increased in CM from ECVL by greater than 10-fold as compared to ECV.

X. Administered Products of Genes

Gene products or proteins identified and administered according to the methods of the invention include TSP-1 and IGFBP-4. Also contemplated for administration are polypeptide portions of IGFBP-4, wherein the portion of the gene product is an active portion having angiogenesis, metastasis or tumor development-inhibiting properties, or it has the ability to exert a beneficial effect on angiogenesis-dependent conditions. IGFBP-4 has been shown to be proteolyzed (see, e.g., Overgaard, J. Biol. Chem. 2000, 275(40):31128-33). It has been reported in the literature that a number of proteins that inhibit angiogenesis, including angiostatin, endostatin, pexstatin, tumstatin, laminin, and fibronectin, have increased anti-angiogenic activity when present in cleaved forms as compared to full-length forms. The resulting cleavage products possess anti-angiogenic activity. For example, the angiogenesis inhibitor, angiostatin, is derived from plasminogen, and the prothrombin kringle-2 domain is a cleavage product of prothrombin (Lee, et al., J. Biol. Chem. 1998, 273 (44):28805-12; Soff, G. A., Cancer Metastasis Rev. 2000, 19(1-2):97-107). A short peptide from matrix metalloproteinase-2 (MMP-2) has also been found to inhibit angiogenesis and tumor growth (U.S. Pub. No. 2002/0182215 A1, incorporated herein by reference in its entirety). Therefore, identified polypeptides, as well as naturally-occurring cleavage products, are contemplated for use according to the methods of the invention. The use of cryptic regions of ECM components having anti-angiogenic function are discussed in, e.g., Schenk, S., et al., Trends in Cell Biol. 2003, 13:366-375 and Kalluri, R. Nat. Rev. Cancer 2003, 3: 422-433.

Gene products can be expressed from genes identified according to the methods of the invention by numerous methods known to those of skill in the art and described in the literature.

For example, recombinantly-produced proteins of the present invention can be directly expressed or expressed as fusion proteins. The recombinant protein can be purified by a combination of cell lysis (e.g., sonication, French press) and affinity chromatography. For fusion products, subsequent digestion of the fusion protein with an appropriate proteolytic enzyme can release the desired recombinant protein.

Polynucleotides containing genes identified using the methods of the present invention may be cloned, using standard cloning and screening techniques, from a cDNA library, (see for instance, Sambrook, et al., Molecular Cloning: A Laboratory Manual, 2nd Ed., Cold Spring Harbor Laboratory Press, Cold Spring Harbor, N.Y. (1989)). These polynucleotides can also be obtained from natural sources such as genomic DNA libraries or can be synthesized using well known and commercially available techniques.

When genes of the present invention are used for the recombinant production of gene products or proteins of the present invention, the polynucleotide including the gene sequence may include the coding sequence for the mature polypeptide, by itself, or the coding sequence for the mature polypeptide in reading frame with other coding sequences, such as those encoding a leader or secretory sequence, a pre-, or pro- or prepro-protein sequence, or other fusion peptide portions. For example, a marker sequence that facilitates purification of the fused polypeptide can be encoded. Polynucleotides can also contain non-coding 5′ and 3′ sequences, such as transcribed, non-translated sequences, splicing and polyadenylation signals, ribosome binding sites and sequences that stabilize mRNA.

There are a number of methods available and well known to those skilled in the art to obtain full-length cDNAs, or extend short cDNAs, for example those based on the method of Rapid Amplification of cDNA ends (RACE) (see, for example, Frohman et al., Proc Nat Acad Sci USA 85, 8998-9002, 1988). Modifications of the technique, exemplified by the Marathon technology (Clontech Laboratories Inc.) for example, have significantly simplified the search for longer cDNAs. In the Marathon technology, cDNAs have been prepared from mRNA extracted from a chosen tissue and an ‘adaptor’ sequence ligated onto each end. Nucleic acid amplification (PCR) is then carried out to amplify the “missing” 5′ end of the cDNA using a combination of gene-specific and adaptor-specific oligonucleotide primers. The PCR reaction is then repeated using ‘nested’ primers, that is, primers designed to anneal within the amplified product (typically an adapter specific primer that anneals further 3′ in the adaptor sequence and a gene specific primer that anneals further 5′ in the known gene sequence). The products of this reaction can then be analyzed by DNA sequencing and a full-length cDNA constructed either by joining the product directly to the existing cDNA to give a complete sequence, or carrying out a separate full-length PCR using the new sequence information for the design of the 5′ primer.

Recombinant polypeptides of the present invention may be prepared by processes well known in the art from genetically engineered host cells comprising expression systems. Accordingly, in a further aspect, the present invention relates to expression systems comprising a polynucleotide or polynucleotides of the present invention, to host cells which are genetically engineered with such expression systems and to the production of polypeptides of the invention by recombinant techniques. Cell-free translation systems can also be employed to produce such proteins using RNAs derived from the DNA constructs of the present invention.

For recombinant production, host cells can be genetically engineered to incorporate expression systems or portions thereof for polynucleotides of the present invention. Polynucleotides may be introduced into host cells by methods described in many standard laboratory manuals, such as Davis et al., Basic Methods in Molecular Biology (1986) and Sambrook et al., 1989. Preferred methods of introducing polynucleotides into host cells include, for instance, calcium phosphate transfection, DEAE-dextran mediated transfection, transfection, micro-injection, cationic lipid-mediated transfection, electroporation, transduction, scrape loading, ballistic introduction or infection.

Representative examples of appropriate hosts include, e.g., bacterial cells, such as Streptococci, Staphylococci, E. coli, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, HEK 293 and Bowes melanoma cells; and plant cells.

As understood in the art, a great variety of expression systems can be used, for instance, chromosomal, episomal and virus-derived systems, e.g., vectors derived from bacterial plasmids, from bacteriophage, from transposons, from yeast episomes, from insertion elements, from yeast chromosomal elements, from viruses such as baculoviruses, papova viruses such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression systems may contain control regions that regulate as well as engender expression. Generally, any system or vector that is able to maintain, propagate or express a polynucleotide to produce a polypeptide in a host may be used. The appropriate polynucleotide sequence may be inserted into an expression system by any of a variety of well-known and routine techniques, such as, for example, those set forth in Sambrook et al., 1989. Appropriate secretion signals may be incorporated into the desired polypeptide to allow secretion of the translated protein into the lumen of the endoplasmic reticulum, the periplasmic space or the extracellular environment. These signals may be endogenous to the polypeptide or they may be heterologous signals.

The proteins of this invention, recombinant or synthetic, can be purified to substantial purity by standard techniques well known in the art, including detergent solubilization, selective precipitation with such substances as ammonium sulfate, column chromatography, immunopurification methods, and others. See, for instance, R. Scopes, Protein Purification: Principles and Practice, Springer-Verlag: New York (1982); Deutscher, Guide to Protein Purification, Academic Press (1990). The protein may then be isolated from cells expressing the protein and further purified by standard protein chemistry techniques.

XI. Methods of Assaying Tumor Metastasis

Tumor metastasis can be measured by a number of techniques known to those of skill in the art and published in the literature. The Examples describe assaying tumor metastasis using the chick embryo model (Brooks et al., Meth. Mol. Biol. 1999, 129:257-269; Testa et al., Cancer Res 1999, 59:3812-3820), and the murine model (Vantyghem, et al., Cancer Res 2003, 63:4763-4765). Subsequent histological and immunofluorescence analyses can be performed as described in the literature (Brooks, et al., Cell 1996, 85:683-693; Brooks, et al., Science 1994, 264:569-571).

XII. Methods of Assaying Angiogenesis

Methods of measuring alterations in angiogenesis are well known in the art. For example, angiogenesis can be measured in the chick chorioallantoic membrane (CAM), in a method referred to as the CAM assay. The CAM assay has been described in detail by others and has been used to measure both angiogenesis and neovascularization of tumor tissues. See Ausprunk et al., Am. J. Pathol., 1975, 79:597-618 and Ossonski et al., Cancer Res. 1980, 40:2300-2309. The CAM assay is a well-recognized assay model for in vivo angiogenesis because it involves the neovascularization of whole tissue with chick embryo blood vessels growing into either the CAM or into the tissue grown on the CAM.

The CAM assay is particularly useful because the system includes an internal control for toxicity. The health of the embryo indicates toxicity since the chick embryo itself is exposed to test reagents.

Another method for measuring alterations in angiogenesis is the in vivo rabbit eye model, referred to as the rabbit eye assay. The rabbit eye assay has been described in detail by others and has been used to measure both angiogenesis and neovascularization in the presence of angiogenic inhibitors such as thalidomide. See D'Amato et al., Proc. Natl. Acad. Sci. 1994, 91:4082-4085.

The rabbit eye assay is a well recognized assay model for in vivo angiogenesis because the neovascularization process, exemplified by rabbit blood vessels growing from the outer rim of the cornea into the cornea, is easily visualized through the naturally transparent corneal membrane. Additionally, both the extent and the amount of stimulation/regression of neovascularization can easily be monitored over time. Finally, this method has an additional benefit of indicating toxicity of the test reagent. Since the rabbit is exposed to test reagents, the health of the rabbit is an indication of toxicity of the test reagent.

Another assay, referred to as the chimeric mouse assay, measures angiogenesis in the chimeric mouse:human mouse model. This assay is described herein, and in detail by others, as a method for measuring angiogenesis, neovascularization, and regression of tumor tissues. See Yan, et al., J. Clin. Invest. 1993, 91:986-996.

The chimeric mouse assay is a useful in vivo model for angiogenesis because the transplanted skin grafts closely resemble normal human skin histologically. Additionally, neovascularization of whole tissue is occurring wherein human blood vessels are growing from grafted human skin into human tumor tissue on the surface of the grafted human skin. The origin of the neovascularization into the human graft can be demonstrated by immunohistochemical staining of the neovasculature with human-specific endothelial cell markers.

The chimeric mouse assay demonstrates regression of neovascularization based on both the amount and extent of new vessel growth. Furthermore, it is easy to monitor effects on the growth of any tissue transplanted upon the grafted skin, such as a tumor tissue. Finally, the assay is useful because there is an internal control for toxicity in the assay system. The health of the mouse is an indication of toxicity when exposed to a test reagent.

To confirm the effects of a compound, e.g., IGFBP-4, on angiogenesis, the mouse Matrigel plug angiogenesis assay can be used. Various growth factors (IGF-1, bFGF or VEGF) (250 ng) and Heparin (0.0025 units per/ml) are mixed with growth factor reduced Matrigel as previously described (Montesano, et al., J. Cell Biol. 1983, 97:1648-1652; Stefansson, et al., J. Biol. Chem. 2000, 276:8135-8141). IGFBP-4 or control BSA (10 to 500 ng) can be included in the Matrigel preparations. In control experiments, Matrigel is prepared in the absence of growth factors. Mice are injected subcutaneously with 0.5 ml of the Matrigel preparation and allowed to incubate for one week. Following the incubation period, the mice are sacrificed and the polymerized Matrigel plugs surgically removed. Angiogenesis within the Matrigel plugs is quantified by two established methods, including immunohistochemical analysis and hemoglobin content (Furstenberger, et al., Lancet. 2002, 3:298-302; Volpert, et al., Cancer Cell 2002, 2(6):473-83.; Su, et al., Cancer Res. 2003, 63:3585-3592). For immunohistochemical analysis, the Matrigel plugs are embedded in OCT, snap frozen and 4 μm sections prepared. Frozen sections are fixed in methanol/acetone (1:1). Frozen sections are stained with polyclonal antibody directed to CD31. Angiogenesis is quantified by microvascular density counts within 20 high powered (200×) microscopic fields.

Hemoglobin content can be quantified as described previously (Schnaper, et al., J. Cell Physiol. 1993, 256:235-246; Montesano, et al., J. Cell Biol. 1983, 97:1648-1652; Stefansson, et al., J. Biol. Chem. 2000, 276:8135-8141; Gigli, et al., J. Immunol. 1986, 100:1154-1164). The Matrigel implants are snap frozen on dry ice and lyophilized overnight. The dried implants are resuspended in 0.4 ml of 1.0% saponin (Calbiochem) for one hour, and disrupted by vigorous pipetting. The preparations are centrifuged at 14,000 g for 15 minutes to remove any particulates. The concentration of hemoglobin in the supernatant is then determined directly by measuring the absorbency at 405 nm and compared to a standard concentration of purified hemoglobin. This method of quantification has been used successfully and has been shown to correlate with angiogenesis (Schnaper, et al., J. Cell Physiol. 1993, 256:235-246; Montesano, et al., J. Cell Biol. 1983, 97:1648-1652; Stefansson, et al., J. Biol. Chem. 2000, 276:8135-8141; Gigli, et al., J. Immunol. 1986, 100:1154-1164).

XIII. Methods of Assaying Cell Adhesion

Cell adhesion can be measured by methods known to those of skill in the art. Assays have been described previously, e.g. by Brooks, et al., J. Clin. Invest 1997, 99:1390-1398. For example, cells can be allowed to adhere to substrate (i.e., an ECM component) on coated wells. Non-attached cells are removed by washing, and non-specific binding sites are blocked by incubation with BSA. The attached cells are stained with crystal violet, and cell adhesion is quantified by measuring the optical density of eluted crystal violet from attached cells at a wavelength of 600 nm.

XIV. Methods of Assaying Cell Migration

Assays for cell migration have been described in the literature, e.g., by Brooks, et al., J. Clin. Invest 1997, 99:1390-1398 and methods for measuring cell migration are known to those of skill in the art. In one method for measuring cell migration described herein, membranes from transwell migration chambers are coated with substrate (here, thermally denatured collagen), the transwells washed, and non-specific binding sites blocked with BSA. Tumor cells from sub-confluent cultures are harvested, washed, and resuspended in migration buffer in the presence or absence of assay antibodies. After the tumor cells are allowed to migrate to the underside of the coated transwell membranes, the cells remaining on the top-side of the membrane are removed and cells that migrate to the under-side are stained with crystal violet. Cell migration is then quantified by direct cell counts per microscopic field.

XV. Methods of Assaying Tumor Growth

Tumor growth can be assayed by methods known to those of skill in the art, e.g., as described in Xu, et al., J. Cell Biol 2001, 154:1069-1079. An assay for chick embryo tumor growth can be performed as follows: single cell suspensions of CS1 melanoma (5×10⁶ per embryo) or HT1080 fibrosarcoma (4×10⁵ per embryo) are applied in a total volume of 40 μl of RPMI to the CAMs of 10-day-old embryos (Brooks et al., 1998). Twenty four hours later, the embryos receive a single intravenous injection of an inhibitor of αvβ3, e.g., LM609, or control molecule (100 μg per embryo). For example, if an antibody inhibitor is used, an isotype-matched antibody can serve as a control. Tumors are grown for 7 days, then resected and wet weights are determined. Experiments can be performed with five to ten embryos per condition.

Another method for assaying tumor growth makes use of the SCID mouse, as follows:

Subconfluent human M21 melanoma cells are harvested, washed, and resuspended in sterile PBS (20×10⁶ per ml). SCID mice are injected subcutaneously with 100 μl of M21 human melanoma cell (2×10⁶) suspension. Three days after tumor cell injection, mice are either untreated or treated intraperitoneally (100 μg/mouse) with either Mab LM609 or an isotype-matched control antibody. The mice are treated daily for 24 days. Tumor size is measured with calipers and the volume estimated using the formula V×L2×W/2, where V is equal to the volume, L is equal to the length, and W is equal to the width.

XVI. Methods of Assaying Cell Proliferation

Cell proliferation can be assayed by methods known to those of skill in the art. As described herein, subconfluent human endothelial cells (HUVECs) can be resuspended in proliferation buffer containing low (5.0%) serum in the presence or absence of CM (25 μl) from ECV or ECVL cells, and endothelial cells allowed to proliferate for 24 hours. Proliferation can be quantified by measuring mitochondrial dehydrogenase activity using a commercially available WST-1 assay kit (Chemicon).

XVII. Methods for Administering Gene Product to a Patient

The dosage ranges for the administration of the product of a gene that is modulated by the specific binding of an antagonist to αvβ3, or fragment thereof, depend upon the form of the gene product, and its potency, and are amounts large enough to produce the desired effect wherein angiogenesis, tumor metastasis, tumor growth, cell adhesion, cell proliferation, or cell migration are inhibited, or wherein the effect is favorable for treatment of an angiogenesis-dependent condition. The dosage should not be so large as to cause adverse side effects, such as hyperviscosity syndromes, pulmonary edema, congestive heart failure, and the like. Generally, the dosage will vary with the age, condition, sex and extent of the disease in the patient and can be determined by one of skill in the art. The dosage can also be adjusted by the individual physician in the event of any complication.

A therapeutically effective amount is an amount of the protein or polypeptide, e.g., a portion of the gene product having angiogenesis-, tumor metastasis-, tumor growth-, cell adhesion- or cell migration-inhibiting properties, sufficient to produce a measurable inhibition of angiogenesis, tumor metastasis, tumor growth, cell adhesion or cell migration in the tissue being treated or to have an effect on an angiogenesis-dependent condition. Inhibition of these symptoms can be measured according to methods described herein, or by other methods known to one skilled in the art. Methods for assessing the effect on an angiogenesis-dependent condition will depend on the condition being treated, and for the particular condition, such methods will be known to those of skill in the art.

It is to be appreciated that the potency, and therefore an expression of a “therapeutically effective” amount can vary. However, as shown by the present assay methods, one skilled in the art can readily assess the potency of a gene product of this invention. Potency can be measured by a variety of means, including, but not limited to: the measurement of inhibition of angiogenesis in the CAM assay, in the in vivo rabbit eye assay, or in the in vivo chimeric mouse:human assay; the inhibition of tumor metastasis in the chick embryo model or in the murine model; the inhibition of cell adhesion in a cell adhesion assay; or the inhibition of cell migration in a cell migration assay, the inhibition of tumor growth in the chick embryo assay or the SCID mouse assay, all as described herein and in the literature and known to those of skill in the art, and the like assays.

A “therapeutically effective” amount of IGFBP-4 can be determined by prevention or amelioration of adverse conditions or symptoms of diseases, injuries or disorders being treated. For all the indications of use of IGFBP-4, the appropriate dosage will of course vary depending upon, for example, the tumor type and stage and severity of the disease disorder to be treated and the mode of administration. For example, tumor inhibition as a single agent may be achieved at a daily dosages from about to 0.1 mg/kg to 40 mg/kg body weight, preferably from about 0.2 mg/kg to about 20 mg/kg body weight of a binding protein of the invention. In larger mammals, for example, humans, as indicated daily dosage is from about 0.25 to about 5 mg/kg/day or about 70 mg per day for an average adult at a dose of 1 mg/kg/day conveniently administered parenterally, for example once a day. Dosage ranges for IGFBP-3 are described in U.S. Publication No. 20040127411, incorporated herein by reference.

The proteins or polypeptides of the invention can be administered parenterally by injection or by gradual infusion over time. Although the tissue to be treated can typically be accessed in the body by systemic administration and therefore most often treated by intravenous administration of therapeutic compositions, other tissues and delivery means are contemplated where there is a likelihood that the tissue targeted contains the target molecule. Thus, proteins or polypeptides of the invention can be administered intravenously, intraperitoneally, intramuscularly, subcutaneously, intracavity, transdermally, and can be delivered by peristaltic means.

Therapeutic compositions are conventionally administered intravenously, as by injection of a unit dose, for example. The term “unit dose” when used in reference to a therapeutic composition of the present invention refers to physically discrete units suitable as unitary dosage for the subject, each unit containing a predetermined quantity of active material calculated to produce the desired therapeutic effect in association with the required diluent, i.e., carrier, or vehicle.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired. Precise amounts of active ingredient required to be administered depend on the judgment of the practitioner and are peculiar to each individual. However, suitable dosage ranges for systemic application are disclosed herein and depend on the route of administration. Suitable regimes for administration are also variable, but are typified by an initial administration followed by repeated doses at one or more hour intervals by a subsequent injection or other administration. Alternatively, continuous intravenous infusion sufficient to maintain concentrations in the blood in the ranges specified for in vivo therapies are contemplated.

The present invention contemplates therapeutic compositions useful for practicing the therapeutic methods described herein. Therapeutic compositions of the present invention contain a physiologically tolerable carrier together with the protein or polypeptide as described herein, dissolved or dispersed therein as an active ingredient. In a preferred embodiment, the therapeutic protein or polypeptide composition is not immunogenic when administered to a mammal or human patient for therapeutic purposes.

As used herein, the terms “pharmaceutically acceptable,” “physiologically tolerable,” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like.

The preparation of a pharmacological composition that contains active ingredients dissolved or dispersed therein is well understood in the art and need not be limited based on formulation. Typically such compositions are prepared as injectables either as liquid solutions or suspensions, however, solid forms suitable for solution, or suspensions in liquid prior to use can also be prepared. The preparation can also be emulsified.

The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient.

The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic, etc. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

Physiologically tolerable carriers are well known in the art. Liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes.

Liquid compositions can also contain liquid phases in addition to and to the exclusion of water. Examples of such additional liquid phases are glycerin, vegetable oils such as cottonseed oil, and water-oil emulsions.

In further embodiments, the invention enables any of the foregoing methods to be carried out in combination with other therapies such as, for example, treatment with another compound, e.g., an inhibitor of angiogenesis and tumor development processes (e.g., a monoclonal antibody that binds to the cryptic collagen epitope, HUIV26), chemotherapy or radiation therapy, or treatment with cytotoxic agents. Chemotherapeutic agents useful in the methods of the present invention include, e.g., taxanes (i.e., Taxol, Docetaxel, Paclitaxel), dacarbazine (DTIC), Adriamycin, Bleomycin, Gemcitabine, Cyclophosphamide, Oxaliplatin, Camptothecan, Ironotecan, Fludarabine, Cisplatin and Carboplatin.

An angiogenesis inhibitor may be administered to a patient in need of such treatment before, during, or after chemotherapy. It is also preferred to administer an angiogenesis inhibitor to a patient as a prophylaxis against metastases after surgery on the patient for the removal of solid tumors.

XVIII. Methods of Detection

Modulation of the expression of IGFBP-4 or TSP-1 can be indicative of the effectiveness of the inhibition of angiogenesis, metastasis, and associated processes resulting from administration of an antagonist that binds to αvβ3.

In the detection methods of the present invention, levels of nucleic acids or proteins can be measured to confirm modulation of the expression levels of IGFBP-4 or TSP-1. Nucleic acid and protein levels can be determined using techniques known to those of skill in the art and described in the literature. For example, nucleic acids can be studied using Real Time quantitative RT-PCR. Primer sequences useful for detecting IGFBP-4 or TSP-1, are given below in the Examples, and additional primer sequences for these genes can be determined by sequence analysis methods and using software as known and commonly used by those of skill in the art.

In addition to PCR techniques, other methods for detecting and quantifying nucleic acids are well known to those skilled in the art and have been described in the literature, including hybridization methods, e.g., Southern blotting, Northern blotting, etc. Amplification techniques, including PCR, can be used prior to analysis using any of these methods.

As described herein, Enzyme Linked Immunosorbent Assay (ELISA) and Western Blot analysis, as well as radioimmunoassay immunoprecipitation, can be used for measuring levels of IGFBP-4 or TSP-1 proteins in the detection methods of the invention.

XIX. Cell Lines

The methods of the present invention can be practiced using a number of cell lines which are obtained and maintained according to methods known to those of skill in the art. For example, murine B16F10 melanoma cell line was obtained from ATCC (Rockville, Md.). Tumor cells were maintained in Dulbecco's Modified Eagles Medium (DMEM) (Gibco Grand Island N.Y.) supplemented with 10% Fetal Bovine Serum (FBS) (Hyclone, Logan Utah), 1.0% Sodium Pyruvate, Glutamate and Pen-Strep (Gibco, Grand Island N.Y.). Cells were maintained as sub-confluent cultures before use and harvested with trypsin-EDTA (Gibco, Grand Island N.Y.).

Cell lines described herein have been previously described, as follows: ECV and ECVL in Brooks, et al., Cell 1998, 92:391-400; M21 and M21L in Montgomery, et al., Proc. Natl. Acad. Sci. USA 1994, 91:8856-8860, and; CS1 and b3CS1 in Brooks, et al., Cell 1996, 85:683-693.

The patents and publications cited herein reflect the level of skill in this field and are hereby incorporated by reference in their entirety to the same extent as if each was specifically and individually indicated to be incorporated by reference.

### EXAMPLES

The present invention is further illustrated by the following examples, which should not be construed as limiting in any way.

### Example I

### Expression of αvβ3 Enhances Tumor Growth In Vivo

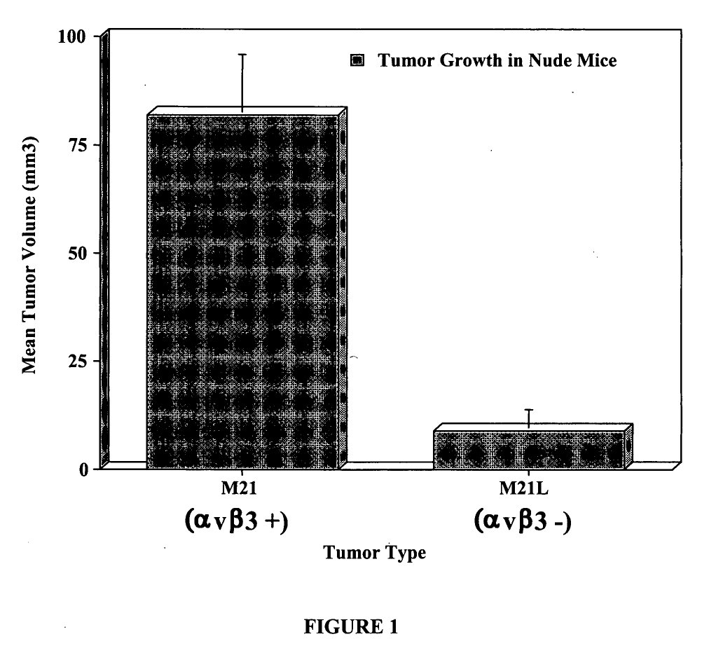

To further investigate the effect of αvβ3 on tumor growth, human melanoma cell variants (M21 or M21L) were injected subcutaneously in nude mice using methods performed similarly to methods previously described (Felding-Habermann, B., Mueller, B. M., Romerdahl, C. A., and Cheresh, D. A. Involvement of integrin αv gene expression in human melanoma tumorigenicity. J. Clin. Invest. 89: 2018-2022 (1992)). Tumor growth was monitored by caliper measurements on day 7. As shown in FIG. 1, αvβ3-expressing M21 cells formed tumors that were approximately 9-fold larger (P<0.05) than tumors from cells that lacked αvβ3 (M21L). These findings confirmed previously reported results and suggest that functional expression of αvβ3 may provide a growth advantage in vivo.

### Example II

### Isolation of αvβ3 Expression Variants of Human ECV304 Bladder Carcinoma Cells

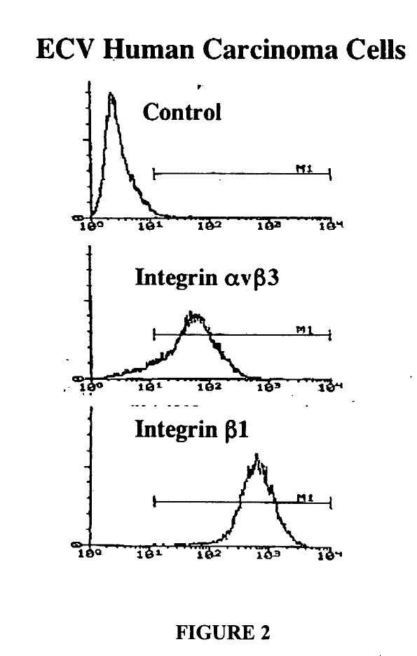

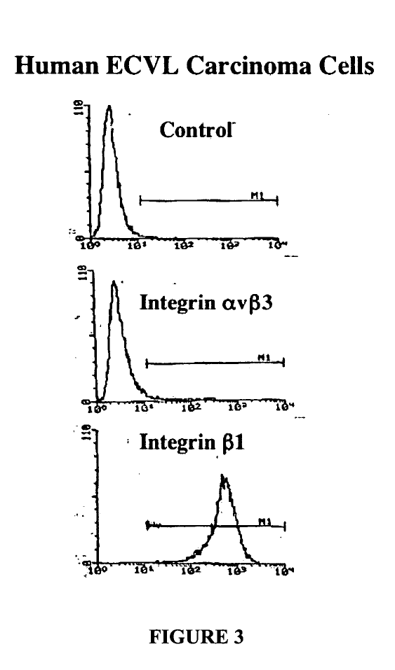

To examine the functional significance of αvβ3 on tumor growth in a histologically distinct tumor type, we isolated variants of the human bladder carcinoma cell line ECV304 that either expressed (ECV) or lacked expression (ECVL) of αvβ3. To isolate these variants, ECV cells were subjected to Fluorescence Activated Cell Sorting (FACS) of cells stained with Mab LM609 directed to αvβ3 integrin. ECV cells were incubated with Mab LM609 and FACS sorted. ECV cells that failed to express cell surface αvβ3 were expanded. The negative FACS selection procedure was carried out a total of 4 times to ensure a stable population of αvβ3 negative ECV cells. As shown in FIG. 2, the parent ECV carcinoma cells expressed high surface levels of αvβ3 (middle panel) and β1 integrins (bottom panel). In contrast, negatively-selected (ECVL) cells (see FIG. 3) expressed no detectable αvβ3 on the cell surface (middle panel). Reduction of αvβ3 expression in these cells resulted in little if any change in β1 integrin expression (bottom panel).

### Example III

### Expression of αvβ3 Enhances Human Carcinoma Growth In Vivo but Not In Vitro

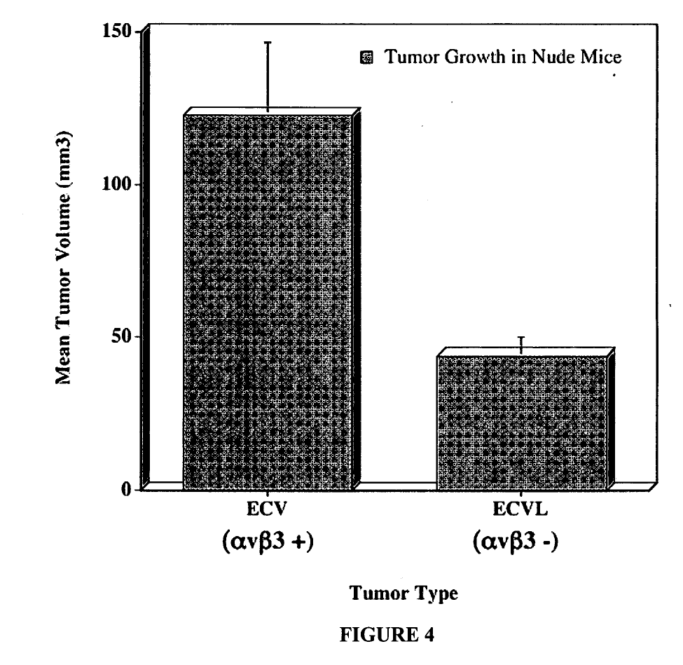

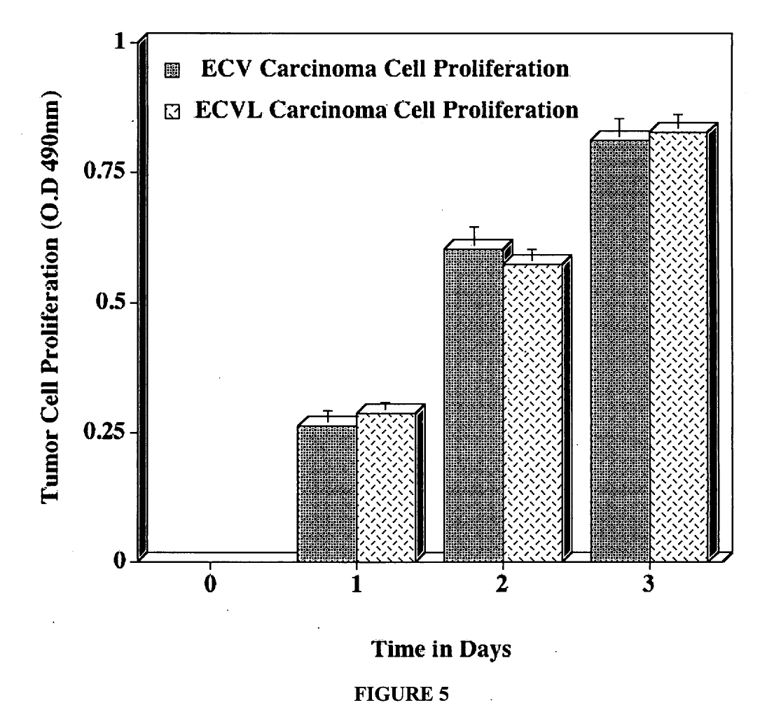

To examine whether loss of αvβ3 cell-surface expression altered tumor growth in vivo, human ECV and ECVL cells were injected subcutaneously in nude mice. Tumor growth was monitored with caliper measurements on day 14 following tumor cell inoculation. As shown in FIG. 4, αvβ3 expressing ECV cells formed tumors that were approximately 3-fold larger than ECVL cells lacking αvβ3. Given the possibility that expression of αvβ3 within ECV cells directly impacts cellular proliferation, thereby contributing to the increase in tumor size, we compared the proliferative capacities of ECV and ECVL cells in vitro. Equal numbers of cells were allowed to proliferation for 3 days. As shown in FIG. 5, little if any change in proliferation was detected between ECV and ECVL cells in vitro. These findings agree with previously published data using M21 and M21L cells (Felding-Habermann, B., Mueller, B. M., Romerdahl, C. A., and Cheresh, D. A. Involvement of integrin αv gene expression in human melanoma tumorigenicity. J. Clin. Invest. 89: 2018-2022 (1992)).

### Example IV

### Elevated Angiogenesis Associated with Tumors Expressing Integrin αvβ3

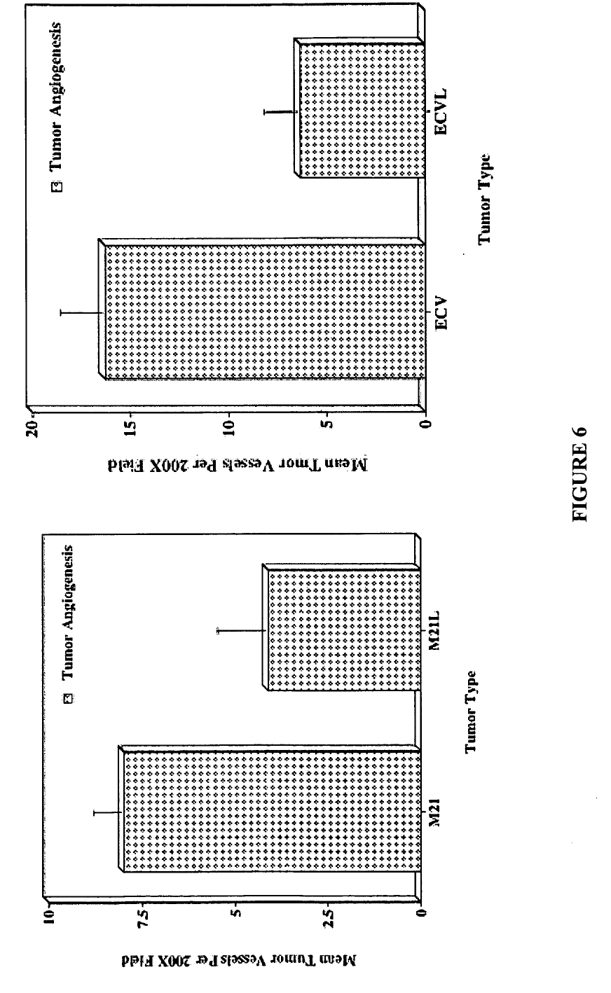

To evaluate the possibility that the growth advantage of αvβ3 expressing tumors (M21 or ECV) may be associated with an increase in angiogenesis, tumors from mice were harvested and tumor angiogenesis was analyzed. Frozen sections of tumors were stained with a polyclonal antibody directed to CD31. The number of CD31-expressing blood vessels per 200× microscopic field were determined using methods previously described (Gasparini, G., Brooks, P. C., Biganzoli, E., Vermeulen, P. B., Bonoldi, E., Dirix, L. Y., Ranieri, G., Miceli, R., and Cheresh, D. A. Vascular integrin alpha (v) beta 3: a new prognostic indicator in breast cancer. Clin. Cancer Res. 11: 2625-2634 (1998)). As shown in FIGS. 6A and 6B, αvβ3-expressing tumors (M21 and ECV) exhibited a significant (P<0.05) 2.0 to 2.5-fold increase in the number of blood vessels as compared to tumors lacking αvβ3 (M21L and ECVL). These findings suggest that αvβ3 modulates angiogenesis within these tumors.

### Example V

### CS1 Melanoma Tumors Expressing αvβ3 Exhibit Enhanced Blood Flow

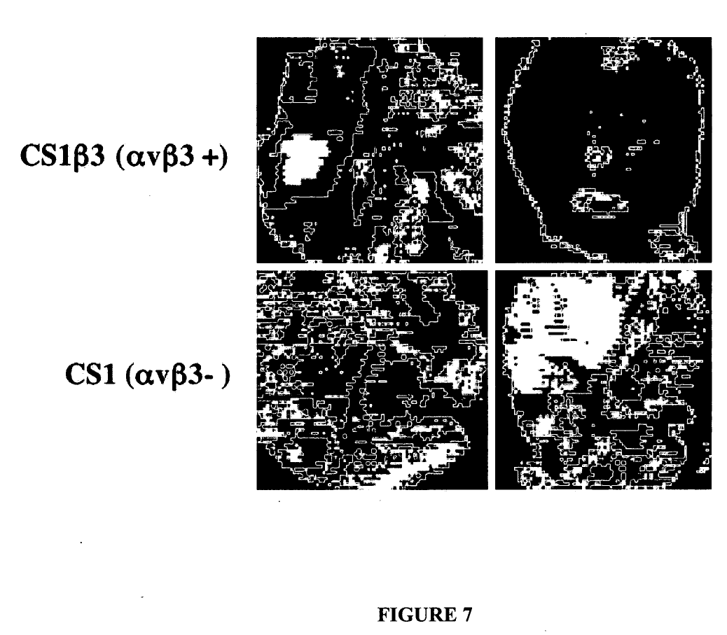

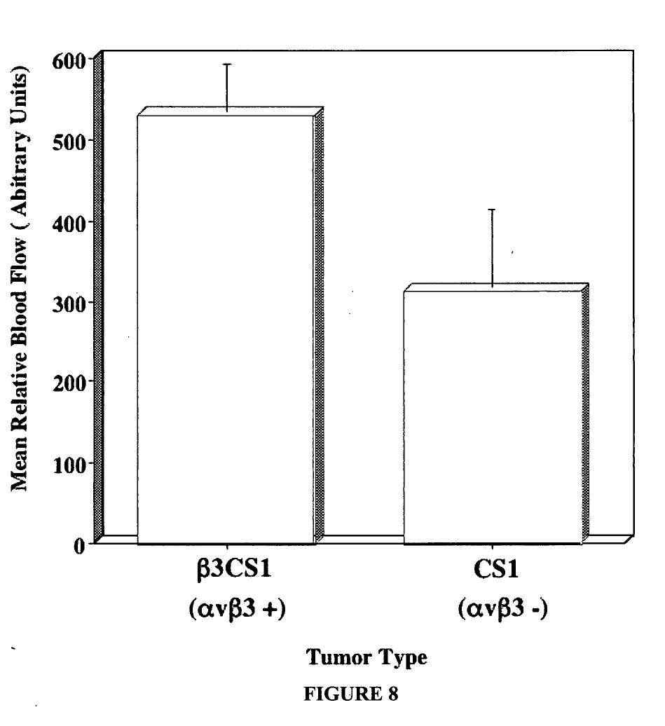

To study the potential role of αvβ3 in tumor angiogenesis in a third model, we examined the relative tumor blood flow in vivo using laser Doppler imaging. CS1 cell variants that either express (CS1β3) or lack (CS1) αvβ3 have been described previously (Brooks, P. C., Klemeke, R. L., Schon, S., Lewis, J. M., Schwartz, M. A., and Cheresh, D. A. Insulin-like growth factor cooperates with integrin alpha v beta 5 to promote tumor cell dissemination in vivo. J. Clin. Invest. 99: 1390-1398 (1997)). CS1 cell variants were inoculated on the CAMs to 10-day old chick embryos (Petitclerc, E., Boutaud, A., Prestayko, A., Xu, J., Sado, Y., Nimomiya, Y., Sarras, M. P., Hudson, B. G., and Brooks, P. C. New Functions for non-collagenous domains of human collagen type-IV: novel integrin ligands inhibiting angiogenesis and tumor growth in vivo. J. Biol. Chem. 275: 8051-8061 (2060)). Tumors were allowed to grow for a total of 7 days and the relative tumor blood flow was examined by laser Doppler scanning (Rai, A., and Gulati, A. Evidence for the involvement of ET(B) receptors in ET-1-induced changes in blood flow to the rat breast tumor. Cancer. Chemother. Pharmacol. 51: 21-28 (2002), Jacob, A., Davis, J. P., and Birchall, M. A. Laser Doppler flux-metry in laryngeal squamous cell carcinoma. Clin. Otolaryngol. 28: 24-28 (2003), and Stanton, A. W. B., Drysdale, S. B., Patel, R., Mellor, R. H., Duff, M. J. B., Levic J. R., and Mortimer, P. S. Expansion of Microvascular bed and increased solute flux in human basal cell carcinoma in vivo, measured by fluorescein video angiography. Cancer Res. 63:3969-3979 (2003)). As shown in FIG. 7, CS103 tumors were associated with elevated levels of blood flow (red color) as compared to CS1 tumors. In fact, CS1β3 tumors were associated with an approximately 40% increase in blood flow as compared to CS1 tumors (P<0.05) that lacked αvβ3 (see FIG. 8).

### Example VI

### Inhibition of Angiogenesis In Vivo by Conditioned Medium (CM) from Tumor Cells Lacking αvβ3

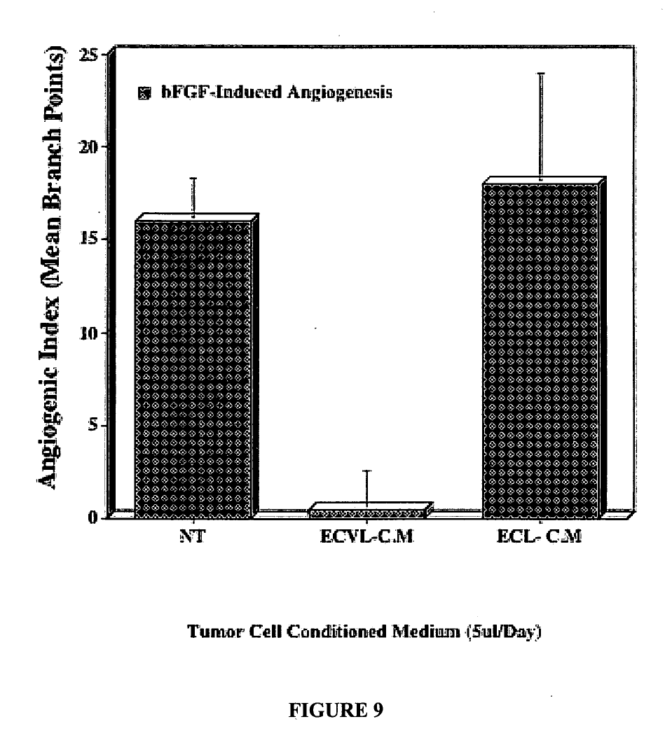

To investigate the possibility that αvβ3 may regulate angiogenesis by modulating expression of angiogenesis inducers, inhibitors, or a combination of both, concentrated serum-free conditioned media (CM) from equal numbers of tumor cells expressing (M21 and ECV) or lacking (M21L and ECVL) αvβ3 were examined for their effects on bFGF-induced angiogenesis. Filter discs containing bFGF were placed on the chorioallantoic membranes (CAMs) of 10-day old chick embryos (Brooks, P. C., Montgomery, A. M., and Cheresh, D. A. Use of the 10-day old chick embryo model for studying angiogenesis. Meth. Mol. Biol. 129: 257-269 (1999)). Twenty-four hours later, the embryos were treated topically (40 ul/day) with CM. At the end of a 3-day incubation period the CAMs were removed and angiogenesis quantified (Brooks, P. C., Montgomery, A. M., and Cheresh, D. A. Use of the 10-day old chick embryo model for studying angiogenesis. Meth. Mol. Biol. 129: 257-269 (1999)). As shown in FIG. 9, CM from ECVL cells significantly (P<0.001) inhibited bFGF-induced angiogenesis by greater than 90% as compared to control. CM from ECV cells had no significant effect (P>0.300) on angiogenesis. In similar studies, CM from M21L cells also (P<0.01) inhibited bFGF-induced angiogenesis by greater than 90%, while CM from M21 cells had only minimal effects on angiogenesis. Taken together, these findings suggest that αvβ3 regulates expression of a secreted inhibitor of angiogenesis.

### Example VII

### Inhibition of Endothelial Cell Proliferation In Vitro by CM from Tumor Cells Lacking αvβ3

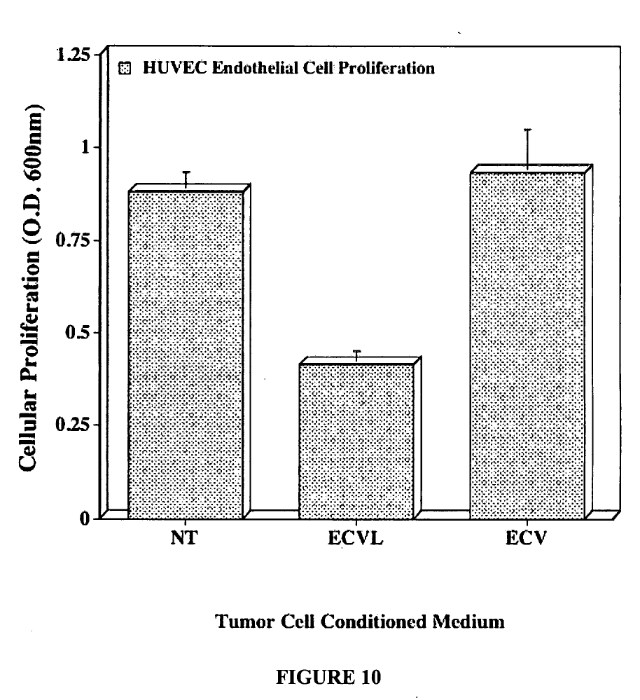

To assess the effects of tumor cell CM on endothelial cell proliferation in vitro, subconfluent human endothelial cells (HUVECs) were resuspendend in proliferation buffer containing low (5.0%) serum in the presence or absence of CM (25 μl) from ECV or ECVL cells. Endothelial cells were allowed to proliferate for 24 hours. Proliferation was quantified by measuring mitochondrial dehydrogenase activity using the commercially available WST-1 assay kit. As shown in FIG. 10, CM from ECVL cells inhibited HUVEC cell proliferation by approximately 50%, while CM from ECV cells had no effect.

### Example VIII

### Inhibition of Tumor Growth In Vivo by CM from Tumor Cells Lacking αvβ3

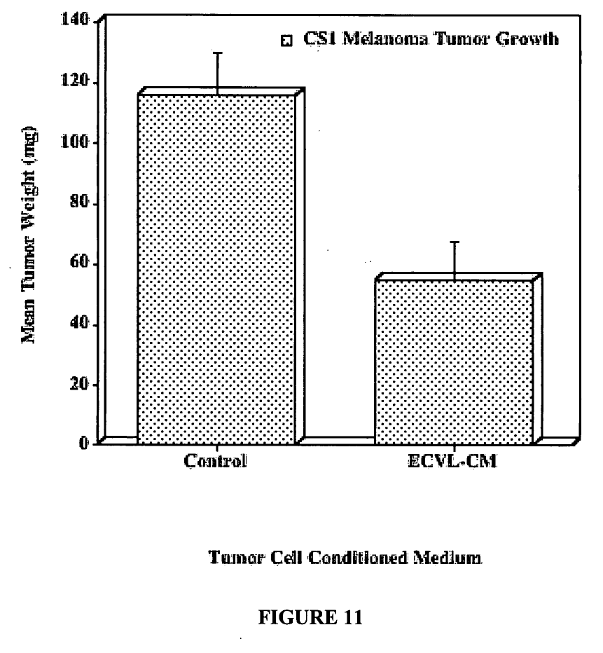

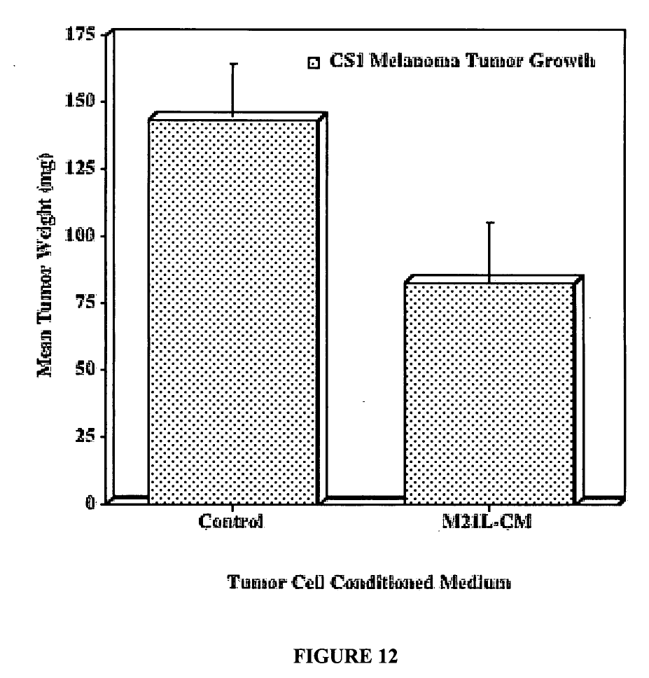

To examine the effects of CM from tumor cells that lacked expression (M21L and ECVL) of αvβ3 on tumor growth in vivo, CS1 tumors were seeded on the CAMs of 10-day old chick (Brooks, P. C., Silletti, S., von Schalscha, T. L., Friedlander, M., and Cheresh, D. A. Disruption of angiogenesis by PEX, a noncatalytic metalloproteinase fragment with integrin binding activity. Cell. 92: 391-400(1998)). The embryos were treated daily by topical addition (25 μl/day) of CM from either M21L or ECVL cells. At the end of a 7-day treatment period the resulting tumors were removed and wet weights determined. As shown in FIGS. 11 and 12, daily treatments with CM from either M21L or ECVL tumor cells resulted in a significant decrease (P<0.05) in tumor weight by approximately 50% as compared to controls.

### Example IX

### Elevation in Levels of TSP-1 in CM from Tumor Cells Lacking αvβ3

To investigate the potential mechanisms by which αvβ3 may regulate angiogenesis, an Affymetrix™-based differential cDNA array analysis was performed on ECV cells that either expressed (ECV) or lacked (ECVL) αvβ3.

To perform the cDNA array analysis, either ECV or ECVL tumor cells were resuspended in serum-free media and added to plates (7×10⁶ cells per plate). The cells were allowed to incubate for a total of 12 hours. Following the 12-hour incubation period, the cells were harvested and the RNA was isolated using both a TRIzol reagent and the Qiagen Rneasy Mini Protocol for RNA Cleanup. After RNA extraction, the amount and quality of RNA was quantified utilizing a spectrophotomer.

5-8 μg of total RNA was utilized to synthesize double-stranded cDNA. The first cDNA strand was obtained using a reaction mixture containing a T7-(dT)24 Primer, 1× First Strand Buffer, 0.1M DTT and 10 mM dNTP mix in addition to the extracted RNA. The tubes were incubated at 42° C. for approximately 1.5 hours. For the second-strand cDNA synthesis, a 1× Second Strand Buffer, 10 mM dNTP mix, 10 U/ml of E. coli DNA Ligase, 10 U/ml of DNA Polymerase I and RNaseH were added and allowed to incubate at 16° C. for 2.5 hours.

Following the incubation period, T4 DNA Polymerase was added and the tubes were incubated for 5 min and stored at −80° C. The final double-stranded cDNA product was cleaned utilizing phenol extraction and ethanol precipitation. Next, the synthesized cDNA was converted to cRNA and labeled with biotin labeled ribonucleotides in a reaction mixture that also included HY Reaction Buffer, 10×DTT, Rnase Inhibitor Mix and 20×RNA Polymerase.

The final cRNA product was cleaned utilizing the Qiagen Rneasy Mini Protocol for RNA Cleanup and 15 μg of cRNA was fragmented and hybridized to a U95Av2 chip.

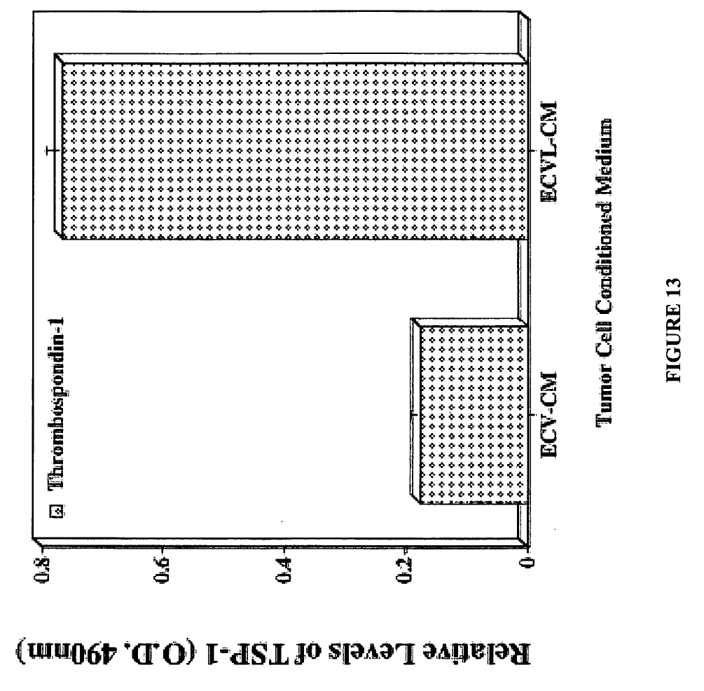

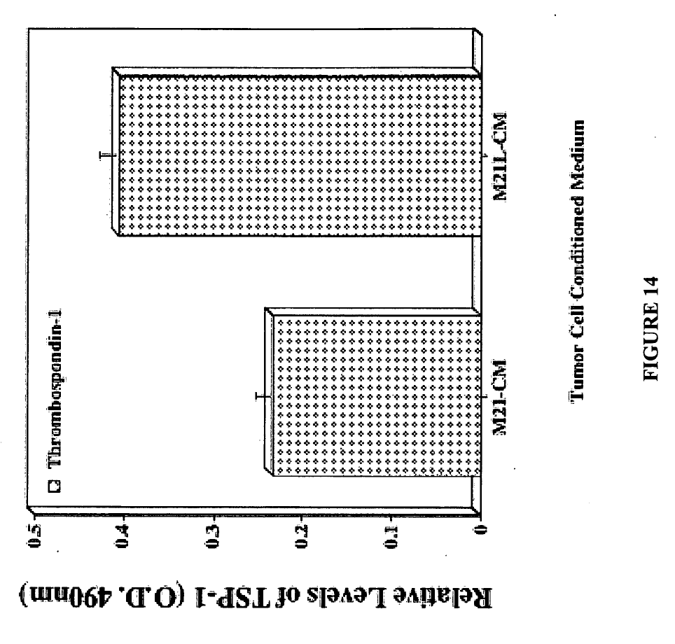

A number of genes within these cells were differentially expressed. Among the genes exhibiting significant upregulation in ECVL as compared to ECV cells, the endogenous angiogenesis inhibitor thrombospondin-1 (TSP-1) was increased by approximately 7-fold. Based on the cDNA array, we analyzed the serum free CM from ECV and M21 cell variants for TSP-1 by solid phase ELISA. As shown in FIGS. 13 and 14, the relative levels of TSP-1 were found to be increased in CM from ECVL and M21L by nearly 2 to 4 fold as compared to CM from ECV and M21.

### Example X

### Reduction of ECVL CM Anti-Proliferative Activity by Immune Depletion of TSP-1

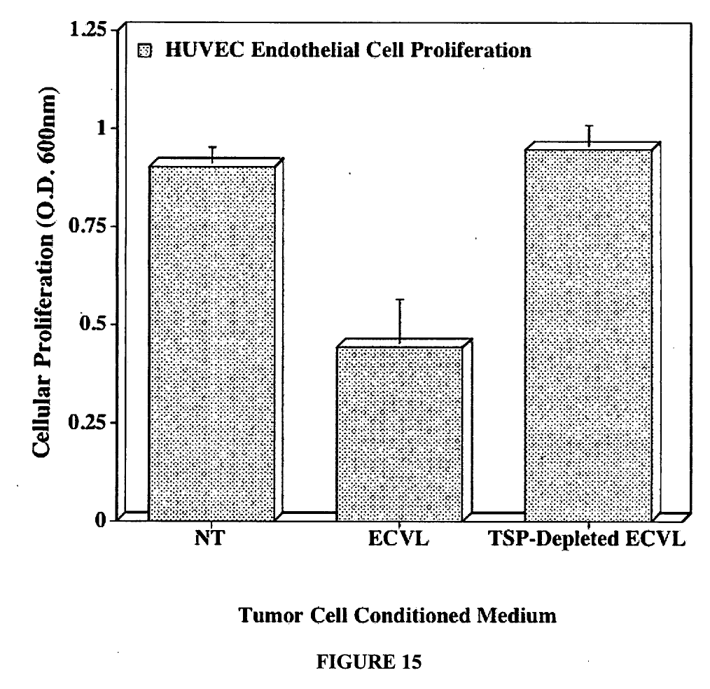

To assess the effects of TSP-1 within the CM of ECVL cells on endothelial cell behavior, we immune depleted TSP-1 from ECVL CM with a Mab directed to TSP-1. CM from ECVL cells was incubated (1 hour) with an anti-TSP-1 Mab followed by incubation with Protein A sepharose beads and the immune complexes were removed by centrifugation. The immune-depletion procedure was carried out 4 times to ensure reduction in TSP-1 levels. The effects of TSP-1 depleted and control depleted ECVL CM on endothelial cell proliferation was carried out as described above. As shown in FIG. 15, control-depleted ECVL conditioned medium inhibited HUVEC proliferation by approximately 50% as compared to no treatment. In contrast, CM from ECVL cells that was depleted of TSP-1 exhibited little if any effects on HUVEC cell proliferation. These data suggest that the presence of elevated levels of TSP-1 in the CM contributes to its ability to inhibit angiogenesis.

### Example XI

### Regulation of IGFBP-4 and TSP-1 by siRNA-Mediated Reduction in β3 Integrin

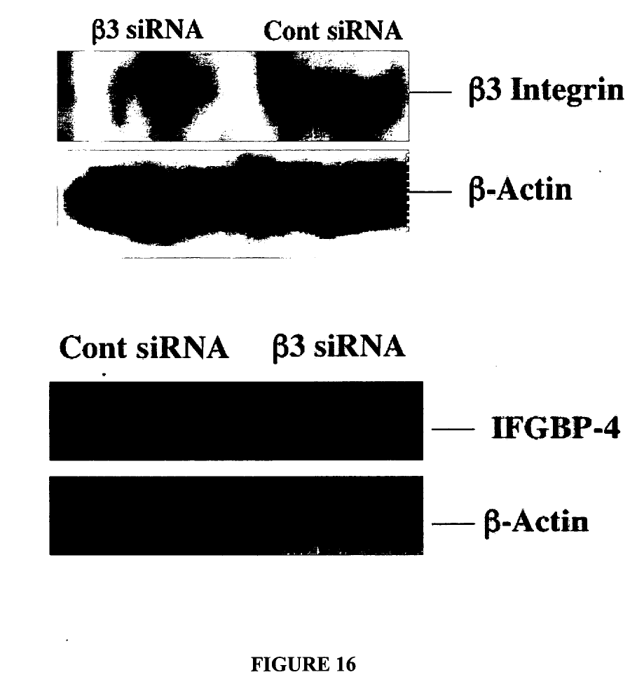

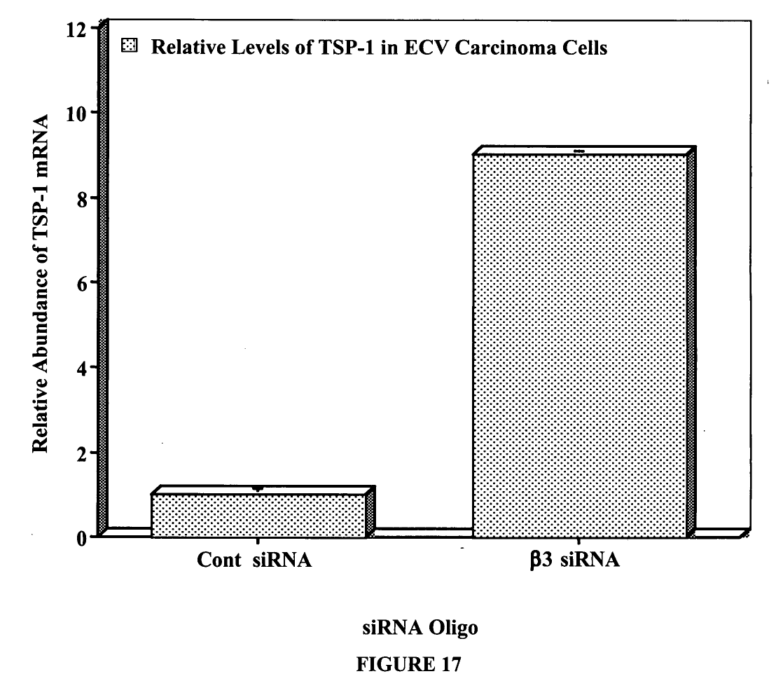

To further study effects of integrin αvβ3 on angiogenesis, we reduced expression of β3 integrin in M21 and ECV cells using siRNA. β3-specific or non-specific siRNA oligos were transfected into tumor cells. Transfectants were isolated, expanded and cell lysates or RNA was prepared for analysis by either Western blot (FIGS. 16A and B) or real time PCR (FIG. 17). As shown in FIG. 16A, β3 integrin was reduced by greater than 70% in β3 siRNA transfected cells as compared to controls, while no change in β-Actin or β1 integrins (data not shown) was observed. In contrast, expression of IGFBP-4 was increased (>60%) in β3 siRNA transfected cells as compared to control cells (FIG. 16B). The relative levels of TSP-1 were significantly elevated in β3 siRNA transfected ECV cells in which β3 integrin is significantly reduced as compared to control transfected cells (FIG. 17). Taken together, these findings suggest that αvβ3 expression and/or ligation may suppress expression of TSP-1 and IGFBP-4.

### Example XII

### αvβ3-Mediated Cellular Interactions Suppress Expression of TSP-1

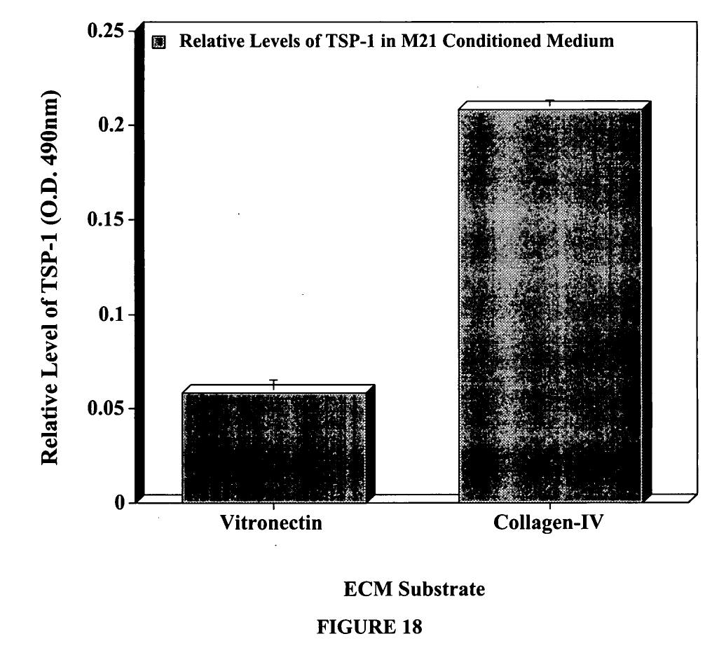

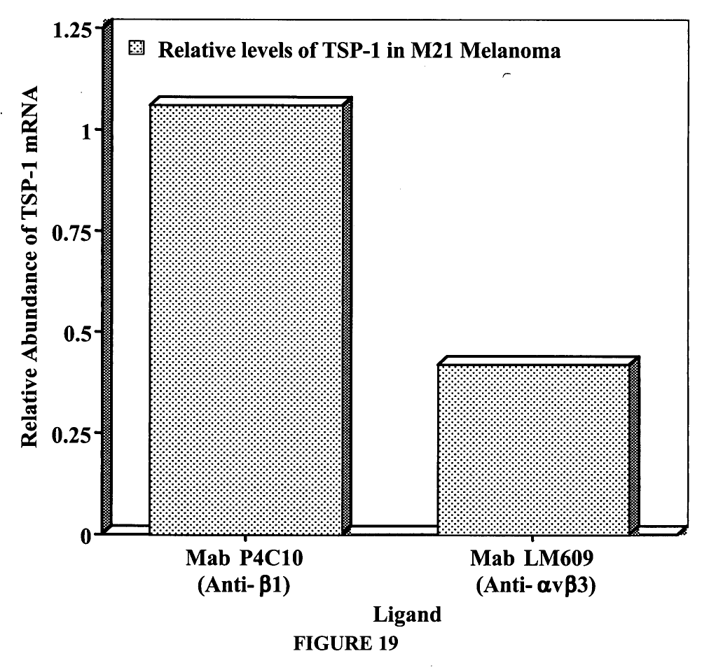

Integrin αvβ3 is known to bind the ECM protein vitronectin but does not bind to triple helical collagen type-IV. Therefore, we assessed the effects of M21 cell interactions with vitronectin on TSP-1 expression in comparison to intact collagen type-IV. M21 cells were allowed to interact with either vitronectin or intact collagen type-IV for 48 hours and the CM was collected and concentrated. The relative level of TSP-1 was assessed within the CM as described above. As shown in FIG. 18, CM from M21 cells interacting with the non-αvβ3 ECM ligand collagen type-IV exhibited an approximately 4 fold increase in TSP-1 as compared to CM from cells interacting with the known αvβ3 ligand vitronectin. Since other αv integrins can also interact with vitronectin, we examined TSP-1 expression in cells specifically interacting with the anti-integrin Mabs known to initiate signaling via distinct integrin receptors (Stromblad, S., Becker, J. C., Yebra, M., Brooks, P. C. and Cheresh, D. A. Suppression of p53 activity and P21WAF1/CIP1 expression by vascular integrin αvβ3 during angiogenesis. J. Clin. Invest. 98: 426-433 (1996); Henriet, P., Zhong, Z. D., Brooks, P. C., Weinberg, K. I., and DeClerk, Y. A. Contact with fibrillar collagen inhibits melanoma cell proliferation by up-regulating p27KIP1. Proc. Natl. Acad. Sci. USA. 97: 10026-10031 (2000)). As shown in FIG. 19, the relative levels of TSP-1 in cells ligating αvβ3 was reduced by greater than 50% as compared to cells ligating β1 integrins as measured by real time PCR. Importantly, the relative levels of TSP-1 were normalized to cells attached to non-integrin ligand (poly-Lysine).

### Example XIII

### Inhibition of αvβ3 Ligation Upregulates TSP-1

To further examine the effect of blocking αvβ3-mediated interactions on TSP-1, we evaluated TSP-1 expression in cells interacting with the known αvβ3 ligand denatured collagen type-IV. Relative expression of TSP-1 was assessed by both real time quantitative RT-PCR and Western Blot analysis. Tumor cells (M21) were allowed to interact with denatured collagen type-IV in the presence or absence of Mab LM609 or an isotype matched control antibody, and mRNA and whole cell lysates were prepared.

Real Time quantitative RT-PCR was carried out essentially as described with some modifications (Livak et al., Method 2001, 25:402-408). Total RNA was isolated using RNeasy miniprep columns (Qiagen, Valencia Calif.) according to the manufacturer's instructions. Total RNA (1 μg) was reverse transcribed using 1× Reverse Transcriptase Buffer, MgCl₂ (3 mM), dNTP (2.0 mM), RNAse inhibitor (0.2 U/μl), random hexamer primers (0.5 mM), and MMLV reverse transcriptase (0.3 U/μl) in 20 μl reactions using a 3-step cycle (Promega, Madison, Wis.). Real-time fluorescence detection was carried out using an ABI Prism 7900 Sequence Detection System. Reactions were carried out in microAmp 96 well reaction plates. Primers and probes were designed using Primer 3 version 2 and ENSEMBL software (Promega).

The following human-specific real time PCR primer pairs were used to detect TSP-1:

| | |
|---|---|
| 5′-TCCAAAGCGTCTTCACCAG-3′ | (SEQ ID NO:1) |
| and | |
| 5′-GAGACAGCCTTTGTTCCTGAG-3′. | (SEQ ID NO:2) |

The primers used to detect control gene β2-macroglobulin were:

| 5′-AAAGATGAGTATGCCTGCCG-3′ | (forward; SEQ ID NO:3) |
|---|---|
| and | |
| 5′-CCTCCATGATGATGCTGCTTACA- | (reverse; SEQ ID NO:4) |
| 3′. | |

cDNA from samples were labeled with SYBR Green (Roche) and real time PCR was run using a Light Cycler (NYU Genomic Core Services). Quantification of data was performed using Light Cycler real time PCR analysis software package 3.5 (Roche).

Fold induction was calculated using methods described by Livak et al, 2001. Amplification products utilized through Sybergreen detection were initially checked by electrophoresis on ethidium bromide stained agarose gels. The estimated size of the amplified products matched the calculated size for transcript by visual inspection.

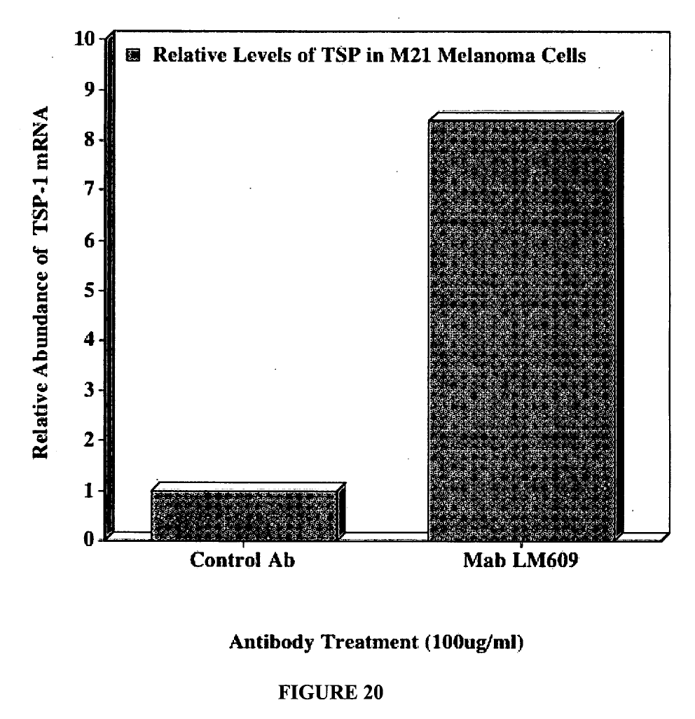

As shown in FIG. 20, the relative level of TSP-1 RNA was elevated by approximately 8-fold in M21 cells treated with the anti-αvβ3 specific Mab LM609 as compared to an isotype matched control antibody as measured by real time PCR. These data suggest that specific inhibition of αvβ3 may increase expression of TSP-1 in vitro and in vivo.

### Example XIV

### Inhibition of αvβ3 Ligation Upregulates IGFBP-4

To examine the effects that blocking αvβ3-mediated interactions has on IGFBP-4 we evaluated the effects of Mab LM609 on IGFBP-4 expression in cells interacting with the known αvβ3 ligand denatured collagen type-IV. M21 cells were allowed to interact with denatured collagen type-IV in the presence or absence of Mab LM609 or an isotype-matched control antibody for 12 hours, and levels of IGFBP-4 RNA were measured by PCR as described with regard to measurement of TSP-1 RNA.

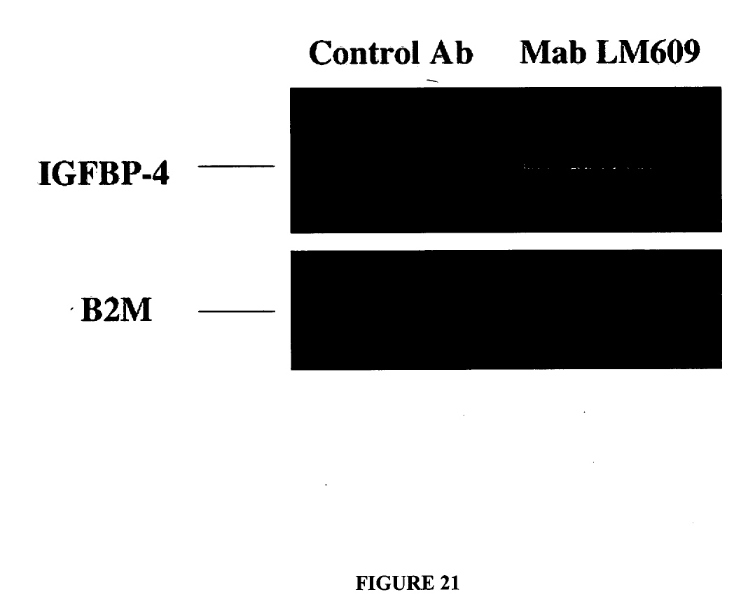

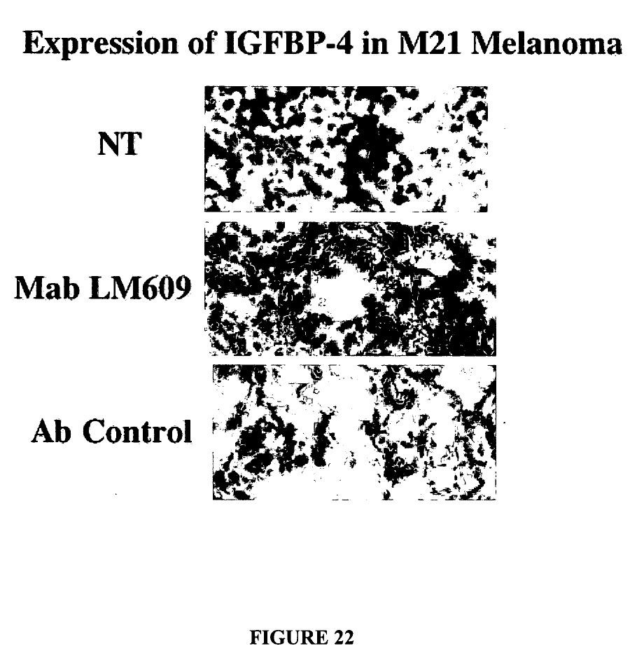

As shown in FIG. 21, the relative level of IGFBP-4 was elevated by approximately 8-fold in M21 cells treated with the anti-αvβ3 specific Mab LM609 as compared to an isotype-matched control antibody. IGFBP-4 RNA was measured by real time PCR as described with regard to measurement of TSP-1 RNA. As shown in FIGS. 21 and 22, expression of IGFBP-4 was significantly enhanced in M21 cells (FIG. 21) and M21 tumors grown in the chick embryo following treatment with Mab LM609 (FIG. 22). These data suggest that specific inhibition of αvβ3 may increase expression of IGFBP-4 in vitro and in vivo.

### Example XV

### Elevated Levels of IGFBP-4 Protein in Conditioned Medium from Tumor Cells Lacking αvβ3

Differential cDNA array analysis suggested increased expression of IFGBP-4 in ECVL as compared to ECV cells. The Affymetrix™-based differential cDNA array analysis was performed similarly to that described in Example IX, comparing ECV and ECVL cells.

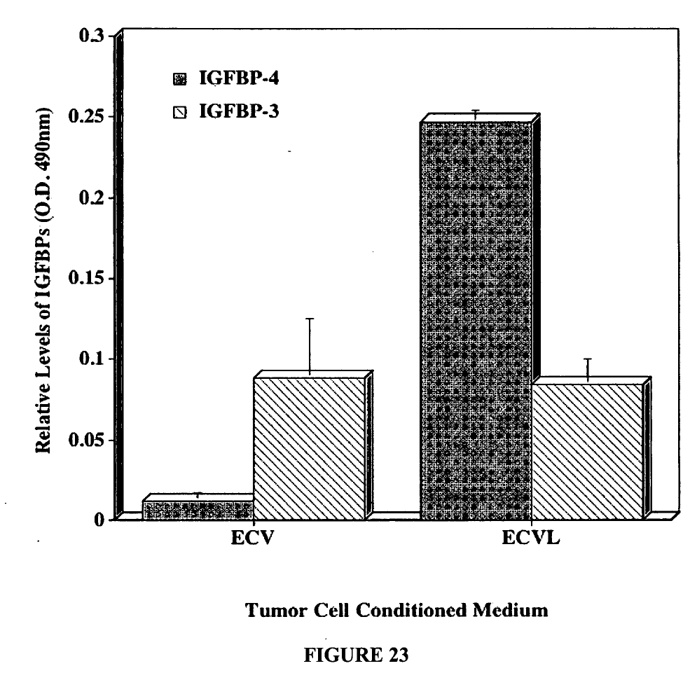

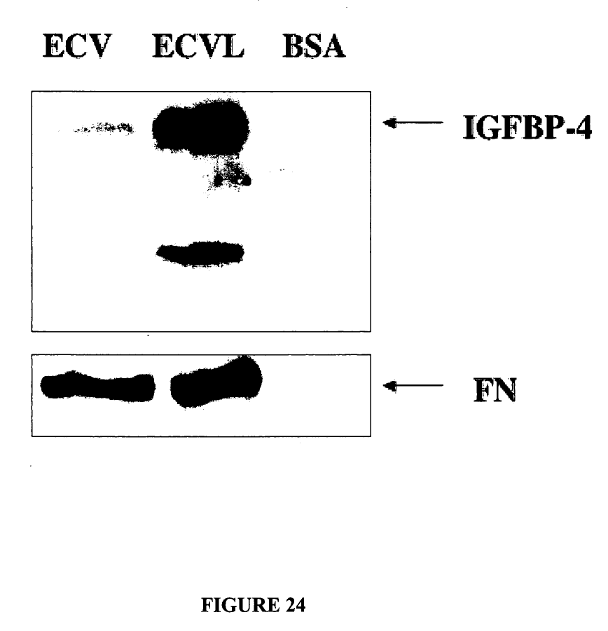

The relative levels of IGFBP-4 were analyzed in conditioned medium (CM) from ECV and ECVL cells by solid phase ELISA (FIG. 23) and Western blot (FIG. 24). As shown by ELISA, the relative levels of IGFBP-4 increased in CM from ECVL by greater than 10-fold as compared to ECV, while little if any change in the levels of IGFBP-3 was observed. As shown in FIG. 24, Western Blot analysis showed that IGFBP-4 was dramatically increased in the CM of ECVL cells as compared to ECV cells while little if any change was detected in soluble fibronectin.

### Example XV

### Peptide Antagonist Inhibition of the Binding of αvβ3 to Tumor Cells Enhances Expression of Certain Genes

To evaluate the effect of blocking αvβ3-mediated interactions using a peptide antagonist, an Affymetrix™-based differential cDNA array analysis is performed using B16F10 tumor cells treated or not treated with the peptide antagonist.

Tumor cells (7×10⁶) are resuspended in serum-containing medium and added to plates in the presence or absence of the peptide antagonist or a control peptide, e.g., as described in U.S. 2003/0176334. The cells are allowed to incubate for a total of 12 hours. Following the 12-hour incubation period, the cells are harvested and the RNA is isolated using both a TRIzol reagent and the Qiagen Rneasy Mini Protocol for RNA Cleanup. After RNA extraction, the amount and quality of RNA is quantified utilizing a spectrophotomer. 5-8 μg of total RNA is utilized to synthesize double-stranded cDNA.

The first cDNA strand is obtained using a reaction mixture containing a T7-(dT)24 Primer, 1× First Strand Buffer, 0.1M DTT and 10 mM dNTP mix in addition to the extracted RNA. The tubes are incubated at 42° C. for approximately 1.5 hours. For the second strand cDNA synthesis, a 1× Second Strand Buffer, 10 mM dNTP mix, 10 U/ml of E. coli DNA Ligase, 10 U/ml of DNA Polymerase I and RNaseH are added and allowed to incubate at 16° C. for 2.5 hours.

Following the incubation period, T4 DNA Polymerase is added and the tubes are again incubated for 5 min and stored at −80° C. The final double-stranded cDNA product is cleaned utilizing phenol extraction and ethanol precipitation. Next, the synthesized cDNA is converted to cRNA and labeled with biotin labeled ribonucleotides in a reaction mixture that also includes HY Reaction Buffer, 10×DTT, Rnase Inhibitor Mix and 20×RNA Polymerase. The final cRNA product is cleaned utilizing the Qiagen Rneasy Mini Protocol for RNA Cleanup and 15 μg of cRNA is fragmented and hybridized to a U95Av2 chip.

Expression levels of differential cDNA array analysis of B16F10 tumor cells treated with the peptide antagonist suggest a significant increase in the expression of certain genes.

Relative expression levels of the genes identified are assessed by both real time quantitative RT-PCR and Western Blot analysis. Tumor cells (B16F10) are allowed to interact with denatured collagen type-IV in the presence or absence of the peptide antagonist or control peptide, and mRNA and whole cell lysates are prepared for use in the analyses.

Real Time quantitative RT-PCR is carried out essentially as described with some modifications (Livak et al., Method 2001, 25:402-408). Total RNA is isolated using RNeasy miniprep columns (Qiagen, Valencia Calif.) according to the manufacturer's instructions. Total RNA (1 μg) is reverse transcribed using 1× Reverse Transcriptase Buffer, MgCl₂ (3 mM), dNTP (2.0 mM), RNAse inhibitor (0.2 U/μl), random hexamer primers (0.5 mM), and MMLV reverse transcriptase (0.3 U/μl) in 20 μl reactions using a 3-step cycle (Promega, Madison, Wis.). Real-time fluorescence detection is carried out using an ABI Prism 7900 Sequence Detection System. Reactions are carried out in microAmp 96 well reaction plates. Primers and probes are designed using Primer 3 version 2 and ENSEMBL software (Promega).

The primers used to detect control gene β2-macroglobulin are:

| 5′-AAAGATGAGTATGCCTGCCG-3′ | (forward; SEQ ID NO:3) |
|---|---|
| and | |
| 5′-CCTCCATGATGATGCTGCTTACA- | (reverse; SEQ ID NO:4) |
| 3′. | |

cDNA from samples is labeled with SYBR Green (Roche) and real time PCR run using a Light Cycler (NYU Genomic Core Services). Quantification of data is performed using Light Cycler real time PCR analysis software package 3.5 (Roche).

Fold induction is calculated using methods described by Livak et al, 2001. Amplification products utilized through Sybergreen detection are initially checked by electrophoresis on ethidium bromide stained agarose gels. The estimated size of the amplified products matches the calculated size for transcript by visual inspection.

### Example XVI

### Organic Peptide Mimetic Antagonist Inhibition of the Binding of αvβ3 to Tumor Cells Enhances Expression of Certain Genes

To evaluate the effect of inhibiting blocking αvβ3-mediated interactions with cryptic epitopes of denatured collagen using an organic peptide mimetic antagonist, an Affymetrix™-based differential cDNA array analysis is performed using B16F10 tumor cells treated or not treated with the organic peptide mimetic antagonist.

Non-tissue culture treated dishes are coated overnight with 100 μg/ml of denatured collagen IV in PBS. The next morning the plates are washed and incubated in blocking solution (1% BSA in PBS) for approximately 30 minutes. Tumor cells (7×10⁶) are resuspended in serum-free media and added to each plate in the presence or absence of the organic peptide mimetic antagonist of αvβ3, e.g., as described in U.S. Pub. No. 2004/0063790, or a control antagonist. The cells are allowed to incubate for a total of 12 hours.

Following the 12-hour incubation period, the cells are harvested and the RNA is isolated using both a TRIzol reagent and the Qiagen Rneasy Mini Protocol for RNA Cleanup. After RNA extraction, the amount and quality of RNA is quantified utilizing a spectrophotomer, and 5-8 μg of total RNA is utilized to synthesize double-stranded cDNA. The first cDNA strand is obtained using a reaction mixture containing a T7-(dT)24 Primer, 1× First Strand Buffer, 0.1M DTT and 10 mM dNTP mix in addition to the extracted RNA. The tubes are incubated at 42° C. for approximately 1.5 hours. For the second strand cDNA synthesis, a 1× Second Strand Buffer, 10 mM dNTP mix, 10 U/ml of E. coli DNA Ligase, 10 U/ml of DNA Polymerase I and RNaseH are added and allowed to incubate at 16° C. for 2.5 hours.

Following the incubation period, T4 DNA Polymerase is added and the tubes are again incubated for 5 min and stored at −80° C. The final double-stranded cDNA product is cleaned utilizing phenol extraction and ethanol precipitation. Next, the synthesized cDNA is converted to cRNA and labeled with biotin labeled ribonucleotides in a reaction mixture that also includes HY Reaction Buffer, 10×DTT, Rnase Inhibitor Mix and 20×RNA Polymerase. The final cRNA product is cleaned utilizing the Qiagen Rneasy Mini Protocol for RNA Cleanup and 15 μg of cRNA is fragmented and hybridized to a U95Av2 chip.

Expression levels of differential cDNA array analysis of B16F10 tumor cells treated with the organic peptide mimetic antagonist suggest a significant increase in the expression of certain genes.

Relative expression levels of the genes identified are assessed by both real time quantitative RT-PCR and Western Blot analysis. Tumor cells (B16F10) are allowed to interact with denatured collagen type-IV in the presence or absence of the organic peptide mimetic antagonist or control antagonist, and mRNA and whole cell lysates are prepared for use in the analyses.

Real Time quantitative RT-PCR is carried out essentially as described with some modifications (Livak et al., Method 2001, 25:402-408). Total RNA is isolated using RNeasy miniprep columns (Qiagen, Valencia Calif.) according to the manufacturer's instructions. Total RNA (1 μg) is reverse transcribed using 1× Reverse Transcriptase Buffer, MgCl₂ (3 mM), dNTP (2.0 mM), RNAse inhibitor (0.2 U/μl), random hexamer primers (0.5 mM), and MMLV reverse transcriptase (0.3 U/μl) in 20 μl reactions using a 3-step cycle (Promega, Madison, Wis.). Real-time fluorescence detection is carried out using an ABI Prism 7900 Sequence Detection System. Reactions are carried out in microAmp 96 well reaction plates. Primers and probes are designed using Primer 3 version 2 and ENSEMBL software (Promega).

The primers used to detect control gene β2-macroglobulin are:

| 5′-AAAGATGAGTATGCCTGCCG-3′ | (forward; SEQ ID NO:3) |
|---|---|
| and | |
| 5′-CCTCCATGATGATGCTGCTTACA- | (reverse; SEQ ID NO:4) |
| 3′. | |

cDNA from samples is labeled with SYBR Green (Roche) and real time PCR run using a Light Cycler (NYU Genomic Core Services). Quantification of data is performed using Light Cycler real time PCR analysis software package 3.5 (Roche).

Fold induction is calculated using methods described by Livak et. al, 2001. Amplification products utilized through Sybergreen detection are initially checked by electrophoresis on ethidium bromide stained agarose gels. The estimated size of the amplified products matches the calculated size for transcript by visual inspection.

What is claimed is:

1. A method for identifying at least one gene or protein, wherein the expression of said gene or protein is modulated by binding of an antagonist to αvβ3, and wherein said antagonist binds to αvβ3 and inhibits binding of αvβ3 to an ECM-component, comprising the steps of:
a) treating cells with the antagonist;
b) measuring gene expression or protein levels in the cells;
c) comparing said gene expression or protein levels with control gene expression or protein levels measured in cells not treated with the antagonist, and;
d) identifying a gene or protein in the cells wherein said gene expression or protein levels in the cells treated with the antagonist are modulated as compared to control cell gene expression or protein levels.

2. The method of claim 1 wherein at least two genes or proteins are identified in the method of identifying, and wherein one of the at least two genes or proteins identified is IGFBP-4 or TSP-1.

3. The method of claim 1 wherein said antagonist is an antibody or an antibody fragment.

4. The method of claim 3 wherein said antibody is a monoclonal antibody or a polyclonal antibody.

5. The method of claim 4 wherein said monoclonal antibody is LM609 (Vitaxin).

6. The method of claim 1 wherein said antagonist is an organic peptidomimetic inhibitor, a peptide or a polypeptide.

7. The method of claim 1 wherein the ECM component is selected from among native collagen, denatured or proteolyzed collagen, native laminin, denatured or proteolyzed laminin, native vitronectin, denatured or proteolyzed vitronectin, native fibrinogen, and denatured or proteolyzed fibrinogen.

8. A method for inhibiting tumor metastasis, cell adhesion, cell migration, tumor growth, cell proliferation, angiogenesis, or for treating an angiogenesis-dependent condition, comprising administering the product of a gene or administering a protein, wherein the gene or the protein is modulated by inhibiting αvβ3, wherein the gene is identified using a method for identifying at least one gene or protein that is modulated by binding of an antagonist to αvβ3, and wherein said antagonist binds to αvβ3 and inhibits binding of αvβ3 to an ECM-component, said method for identifying comprising the steps of:
a) treating cells with the antagonist;
b) measuring gene expression or protein levels in the cells;
c) comparing said gene expression or protein levels with control gene expression or protein levels measured in cells not treated with the antagonist, and;
d) identifying a gene or protein in the cells wherein said gene expression or protein levels in the cells treated with the antagonist are modulated as compared to control cell gene expression or protein levels.

9. The method of claim 8 wherein said gene product or protein is administered in conjunction with chemotherapy, radiation therapy, or a cytostatic agent.

10. The method of claim 8 wherein at least two genes or proteins are identified in the method of identifying, and wherein one of the at least two genes or proteins identified is IGFBP-4 or TSP-1.

11. The method of claim 8 wherein said antagonist is an antibody or an antibody fragment.

12. The method of claim 11 wherein said antibody is a monoclonal antibody or a polyclonal antibody.

13. The method of claim 12 wherein said monoclonal antibody is LM609 (Vitaxin®).

14. The method of claim 8 wherein said antagonist is an organic peptidomimetic inhibitor, a peptide or a polypeptide.

15. The method of claim 8 wherein the ECM component is selected from among native collagen, denatured or proteolyzed collagen, native laminin, denatured or proteolyzed laminin, native vitronectin, denatured or proteolyzed vitronectin, native fibrinogen, and denatured or proteolyzed fibrinogen.

16. An antagonist that binds to αvβ3, wherein binding of said antagonist inhibits the binding of αvβ3 to an ECM component, and wherein the binding of said antagonist to said ECM component results in modulation of IGFBP-4 or TSP-1.

17. The antagonist of claim 16, wherein said antagonist is an antibody or an antibody fragment.

18. The antagonist of claim 17, wherein said antibody is a monoclonal antibody or a polyclonal antibody.

19. The antagonist of claim 18, wherein said monoclonal antibody is LM609 (Vitaxin®).

20. The antagonist of claim 16, wherein said antagonist is an organic peptidomimetic inhibitor, a peptide or a polypeptide.

21. The antagonist of claim 16, wherein the ECM component is selected from among native collagen, denatured or proteolyzed collagen, native laminin, denatured or proteolyzed laminin, native vitronectin, denatured or proteolyzed vitronectin, native fibrinogen, and denatured or proteolyzed fibrinogen.

22. A method for inhibiting tumor metastasis, cell adhesion, cell migration, tumor growth, angiogenesis, cell proliferation, or for treating an angiogenesis-dependent condition comprising administering an antagonist of claim 16.

23. The method of claim 22 wherein said gene product or protein is administered in conjunction with chemotherapy, radiation therapy, or with a cytostatic agent.

24. A method of detecting the inhibition of tumor metastasis, cell adhesion, cell migration, tumor growth, angiogenesis, or cell proliferation by administering an antagonist that specifically binds αvβ3, comprising: measuring the level of IGFBP-4 or TSP-1, wherein said level of IGFBP-4 or TSP-1 is modulated.


SEQUENCE LISTING (4 sequences)

SEQ ID NO: 1 - DNA, 19 bp, Artificial
  PCR primer
tccaaagcgtcttcaccag

SEQ ID NO: 2 - DNA, 21 bp, Artificial
  PCR primer
gagacagcctttgttcctgag

SEQ ID NO: 3 - DNA, 20 bp, Artificial
  PCR primer
aaagatgagtatgcctgccg

SEQ ID NO: 4 - DNA, 23 bp, Artificial
  PCR primer
cctccatgatgatgctgcttaca